SEQUENCE LISTING (51 sequences)

SEQ ID NO: 1 - protein, 482 aa, Pseudomonas putida
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 2 - protein, 432 aa, Artificial Sequence
  source: /note=“Description of Artificial Sequence Synthetic polypeptide”
GFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 3 - protein, 457 aa, Artificial Sequence
  source: /note=“Description of Artificial Sequence Synthetic polypeptide”
GVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 4 - protein, 444 aa, Artificial Sequence
  source: /note=“Description of Artificial Sequence Synthetic polypeptide”
TQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 5 - protein, 481 aa, Artificial Sequence
  source: /note=“Description of Artificial Sequence Synthetic polypeptide”
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGRWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 6 - protein, 481 aa, Artificial Sequence
  source: /note=“Description of Artificial Sequence Synthetic polypeptide”
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGKWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 7 - protein, 481 aa, Artificial Sequence
  source: /note=“Description of Artificial Sequence Synthetic polypeptide”
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 8 - protein, 432 aa, Artificial Sequence
  source: /note=“Description of Artificial Sequence Synthetic polypeptide”
GFDYDVVVVGGGFAGATAARECGLQGYRTLLLEARSRLGGRTFTSRFAGQEIEFGGRWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 9 - protein, 481 aa, Artificial Sequence
  source: /note=“Description of Artificial Sequence Synthetic polypeptide”
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGHWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 10 - protein, 481 aa, Artificial Sequence
  source: /note=“Description of Artificial Sequence Synthetic polypeptide”
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGPWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 11 - protein, 447 aa, Artificial Sequence
  source: /note=“Description of Artificial Sequence Synthetic polypeptide”
QVRLQESGPGLVKPSGTLSLTCAVSGGSIYSSNWWTWVRQPPGKGLEWVGEIHIRGTTYYNPSLNSRVTISLDKSNNQVSLRLTSVTAADSAVYYCVSQEVGGPDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  source: /note=“Description of Artificial Sequence Synthetic polypeptide”
NFMLTQPHSVSESPGKTVTISCTRSGGSIATYYVQWYQQRPGSAPTNVIYKYDQRPSGVPDRFSGSIDSSSNSASLTISGLKTEDEADYYCQSYDNNIQVFGGGTKLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 453 aa, Artificial Sequence
  source: /note=“Description of Artificial Sequence Synthetic polypeptide”
QLQLQESGPGLVKPSETLSLICTVSGGSIRKNNEWWAWIRQAPGKGLEWIGSLSYTGRTVYNPSLKSRVTISTDTSETQFSLKVNSVTAADTAVYYCARLSPFVGAAWWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Artificial Sequence
  source: /note=“Description of Artificial Sequence Synthetic polypeptide”
EVVLTQSPGTLSLSPGERATLSCRASQSVSSRYLAWYQQKPGQAPRLLIYGASSRAIGTPDRFSGSGSGTDFTLTISRLEPEDFAVYYCQQYAYSPPAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 456 aa, Artificial Sequence
  source: /note=“Description of Artificial Sequence Synthetic polypeptide”
QLQLQESGPGLLKPSETLSLTCTVSGGSVTTSPDWWAWLRQSPGKGLEWIGSVSYTGRTVYNPSLKSRVTISLDTSKNHLSLRMTSATAADTAVFYCARLTPIDRFSADYYVLDIWGQGATVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 216 aa, Artificial Sequence
  source: /note=“Description of Artificial Sequence Synthetic polypeptide”
EIVMTQSPATLSVSPGERATLSCRASQSISSNLAWFQHKPGQAPRLLIFRSSTRATGTPPRFSGSGSGTEFTLTISSLQSEDFAVYFCQHYSYWPPLITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 458 aa, Artificial Sequence
  source: /note=“Description of Artificial Sequence Synthetic polypeptide”
QLQLRESGPGLVKPSETLSLTCSVSGGSISSSSYYWGWIRQPPGKGLEWIGSIYYTGRTYYNPSLESRVTISVDTSKNQFSLKLSSVTAADTAVYYCAGLHYSWSALGGYYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 217 aa, Artificial Sequence
  source: /note=“Description of Artificial Sequence Synthetic polypeptide”
EIVLTQSPGTLSLSPGERATLSCRASQSVSSRDLVWYQQKPGQAPRLLIYGASTRATGIPDRFSGSGSGTDFTLTISRLEPEDFAVYYCQKYGSSPPRITFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 452 aa, Artificial Sequence
  source: /note=“Description of Artificial Sequence Synthetic polypeptide”
QLQLQESGPGLVKPSETLSLTCSVSGGSISSSSYYWGWSRQSPGKGLEWIASIYYSGSTYYNPSLKSRVTIFIDTSKNQFSLKLSSVTAADTAIYYCARVGTSAMSRAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, Artificial Sequence
  source: /note=“Description of Artificial Sequence Synthetic polypeptide”
DIVMTQSPLSLPVTPGEPASISCRSSQSLLQSNGYNYLDWYLQKPGQSPQLLIYLGSNRASGVPDRFSGSGSGTDFTLKISKVEAEDVGVYFCMQALQIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 453 aa, Artificial Sequence
  source: /note=“Description of Artificial Sequence Synthetic polypeptide”
QVQLQESGPGLVKPSETLSLTCTVSGGSISRRNDYWAWIRQSPGKDLEWIGTISFSGSTFYNPSLKSRVTISADTFNNHFSLRLDAVAAADTAVYYCARLSPFVGAAWWFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 217 aa, Artificial Sequence
  source: /note=“Description of Artificial Sequence Synthetic polypeptide”
EIVLTQSPGTLSLSPGERATLSCRASQSLSSNYLGWYQQKPGQAPRLLIYGASNRATGIPDRFSGSGSGTDFTLTISRLEPEDFGVYYCQRYGRSPPAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 457 aa, Artificial Sequence
  source: /note=“Description of Artificial Sequence Synthetic polypeptide”
QLQLQESGPGLVKPSETLSLTCTASGGSITNNIDYWVWIRQPPGRGLEWIGTIYYSGSTFYNPSLKSRVTISVDTSNNQFSLNLNSMSAADTAVYYCARLRYYYDSNGYLPYWIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 217 aa, Artificial Sequence
  source: /note=“Description of Artificial Sequence Synthetic polypeptide”
EIVLTQSPGTLSLSPGERATLSCRASQSISSSYLGWYQQKPGQAPRLLIYGASSRATGIPDRFSGSGSGTDFTLTISSLEPEDFAVYFCQLYRRSPPRLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 455 aa, Artificial Sequence
  source: /note=“Description of Artificial Sequence Synthetic polypeptide”
QLQLQESGPGLVKPSESLSLTCTVSGGSIISNDYYWAWIRQSPGKGLEWIGSINYRGSTFYSPSLNSRVTTSVDTSKNQFFLKLTSVTAADTAMYFCTRLHGRYRGVGRLAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  source: /note=“Description of Artificial Sequence Synthetic polypeptide”
DIQMTQSPSTLSASVGDIVTITCRASQSIGDWLAWYQQKPGKAPKLLIYKASNLESGVPSRFSGSGSGTEFTLTISSLQSDDFATYYCQQYDSYSVTFGQGTKVEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 456 aa, Artificial Sequence
  source: /note=“Description of Artificial Sequence Synthetic polypeptide”
QVQLQEAGPGLVKPSETLSLTCTVSGGSINTRNYYWGWVRQPPGKGLEWIASVYYTGSTFYDPSLRSRVTISIDTPRNQFSLRVSSVDAGDMGVYYCVRLDGGYNNGYYYYGMDVWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Artificial Sequence
  source: /note=“Description of Artificial Sequence Synthetic polypeptide”
GVQMTQSPSTLSASVGERVTVTCRASRPISNWLSWYQQKPGRAPKLLIYGTSTLESGVPSRFSGSGSGTEFTLTITNLQPDDFATYYCQEHNLYTITFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 457 aa, Artificial Sequence
  source: /note=“Description of Artificial Sequence Synthetic polypeptide”
QLQLQESGPGLVKPSETLSLSCAVSGASIRSNTYYWGWIRQPPGRGLEWIGSISHRGDAHYSPSLKSPVTISVDTSKNEFSLKATSVTAADTAVYYCVSLAYSFSWNTYYFYGMDVWGHGITVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 217 aa, Artificial Sequence
  source: /note=“Description of Artificial Sequence Synthetic polypeptide”
DIVLTQSPGTLSLSPGEGATLSCRASQSVNSGYLAWYQQKPGQPPRLLVFAASSRATGIADRFRGSGSGTDFTLTITRLEPEDFAVYYCQLYGHSPARITFGQGTRLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 449 aa, Artificial Sequence
  source: /note=“Description of Artificial Sequence Synthetic polypeptide”
QLQLQESGPGLVKPSETLSLTCSVSGGSISSSSYYWGWSRQSPGKGLEWIASIYYSGSTYYNPSLKSRVTIFIDTSKNQFSLKLSSVTAADTAIYYCARVGTSAMSRAFDMWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 219 aa, Artificial Sequence; identical to SEQ ID NO: 20
  source: /note=“Description of Artificial Sequence Synthetic polypeptide”

SEQ ID NO: 33 - protein, 453 aa, Artificial Sequence
  source: /note=“Description of Artificial Sequence Synthetic polypeptide”
QLQLQESGPGLLKPSETLSLTCTVSGGSVTTSPDWWAWLRQSPGKGLEWIGSVSYTGRTVYNPSLKSRVTISLDTSKNHLSLRMTSATAADTAVFYCARLTPIDRFSADYYVLDIWGQGATVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 216 aa, Artificial Sequence; identical to SEQ ID NO: 16
  source: /note=“Description of Artificial Sequence Synthetic polypeptide”

SEQ ID NO: 35 - protein, 450 aa, Artificial Sequence
  source: /note=“Description of Artificial Sequence Synthetic polypeptide”
QLQLQESGPGLVKPSETLSLICTVSGGSIRKNNEWWAWIRQAPGKGLEWIGSLSYTGRTVYNPSLKSRVTISTDTSETQFSLKVNSVTAADTAVYYCARLSPFVGAAWWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 217 aa, Artificial Sequence; identical to SEQ ID NO: 14
  source: /note=“Description of Artificial Sequence Synthetic polypeptide”

SEQ ID NO: 37 - protein, 444 aa, Artificial Sequence
  source: /note=“Description of Artificial Sequence Synthetic polypeptide”
QVRLQESGPGLVKPSGTLSLTCAVSGGSIYSSNWWTWVRQPPGKGLEWVGEIHIRGTTYYNPSLNSRVTISLDKSNNQVSLRLTSVTAADSAVYYCVSQEVGGPDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 216 aa, Artificial Sequence; identical to SEQ ID NO: 12
  source: /note=“Description of Artificial Sequence Synthetic polypeptide”

SEQ ID NO: 39 - protein, 455 aa, Artificial Sequence
  source: /note=“Description of Artificial Sequence Synthetic polypeptide”
QLQLRESGPGLVKPSETLSLTCSVSGGSISSSSYYWGWIRQPPGKGLEWIGSIYYTGRTYYNPSLESRVTISVDTSKNQFSLKLSSVTAADTAVYYCAGLHYSWSALGGYYFYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 217 aa, Artificial Sequence; identical to SEQ ID NO: 18
  source: /note=“Description of Artificial Sequence Synthetic polypeptide”

SEQ ID NO: 41 - protein, 450 aa, Artificial Sequence
  source: /note=“Description of Artificial Sequence Synthetic polypeptide”
QVQLQESGPGLVKPSETLSLTCTVSGGSISRRNDYWAWIRQSPGKDLEWIGTISFSGSTFYNPSLKSRVTISADTFNNHFSLRLDAVAAADTAVYYCARLSPFVGAAWWFDPWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 217 aa, Artificial Sequence; identical to SEQ ID NO: 22
  source: /note=“Description of Artificial Sequence Synthetic polypeptide”

SEQ ID NO: 43 - protein, 454 aa, Artificial Sequence
  source: /note=“Description of Artificial Sequence Synthetic polypeptide”
QLQLQESGPGLVKPSETLSLTCTASGGSITNNIDYWVWIRQPPGRGLEWIGTIYYSGSTFYNPSLKSRVTISVDTSNNQFSLNLNSMSAADTAVYYCARLRYYYDSNGYLPYWIDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 217 aa, Artificial Sequence; identical to SEQ ID NO: 24
  source: /note=“Description of Artificial Sequence Synthetic polypeptide”

SEQ ID NO: 45 - protein, 452 aa, Artificial Sequence
  source: /note=“Description of Artificial Sequence Synthetic polypeptide”
QLQLQESGPGLVKPSESLSLTCTVSGGSIISNDYYWAWIRQSPGKGLEWIGSINYRGSTFYSPSLNSRVTTSVDTSKNQFFLKLTSVTAADTAMYFCTRLHGRYRGVGRLAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 214 aa, Artificial Sequence; identical to SEQ ID NO: 26
  source: /note=“Description of Artificial Sequence Synthetic polypeptide”

SEQ ID NO: 47 - protein, 453 aa, Artificial Sequence
  source: /note=“Description of Artificial Sequence Synthetic polypeptide”
QVQLQEAGPGLVKPSETLSLTCTVSGGSINTRNYYWGWVRQPPGKGLEWIASVYYTGSTFYDPSLRSRVTISIDTPRNQFSLRVSSVDAGDMGVYYCVRLDGGYNNGYYYYGMDVWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 214 aa, Artificial Sequence; identical to SEQ ID NO: 28
  source: /note=“Description of Artificial Sequence Synthetic polypeptide”

SEQ ID NO: 49 - protein, 454 aa, Artificial Sequence
  source: /note=“Description of Artificial Sequence Synthetic polypeptide”
QLQLQESGPGLVKPSETLSLSCAVSGASIRSNTYYWGWIRQPPGRGLEWIGSISHRGDAHYSPSLKSPVTISVDTSKNEFSLKATSVTAADTAVYYCVSLAYSFSWNTYYFYGMDVWGHGITVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 217 aa, Artificial Sequence; identical to SEQ ID NO: 30
  source: /note=“Description of Artificial Sequence Synthetic polypeptide”

SEQ ID NO: 51 - protein, 14 aa, Artificial Sequence
  source: /note=“Description of Artificial Sequence Synthetic peptide”
GGGGSGSGHHHHHH

(19) United States
(12) Patent Application Publication
(10) Pub. No.: US 2022/0267474 A1
(43) Pub. Date: Aug. 25, 2022

(54) METHODS FOR IMPROVING CIRCULATION AND TREATING CARDIOVASCULAR DISEASE

(71) Applicant: ANTIDOTE THERAPEUTICS, INC., Bethesda, MD (US)

(72) Inventors: Matthew W. KALNIK, Bethesda, MD (US); Thomas THISTED, New Market, MD (US); Warren C. STERN, Hilton Head Island, SC (US)

(73) Assignee: ANTIDOTE THERAPEUTICS, INC., Bethesda, MD (US)

(21) Appl. No.: 16/972,060

(22) PCT Filed: Jun. 5, 2019
(86) PCT No.: PCT/US2019/035512

§ 371 Date: Dec. 4, 2020

Related U.S. Application Data
(60) Provisional application No. 62/681,342, filed on Jun. 6, 2018.

(51) Int. Cl. C07K 16/44; A61P 25/34

(52) U.S. Cl. CPC C07K 16/44; A61P 25/34

(57) ABSTRACT

Described are methods of treating cardiovascular diseases in smokers and/or subjects ingesting or consuming nicotine from other sources (e-cigarettes/vaping, smokeless tobacco, NRT, etc.) by using anti-nicotine agents such as nicotine-binding antibodies or nicotine-degrading enzymes, as well as anti-nicotine agents for use in such

### CROSS-REFERENCE TO RELATED APPLICATIONS

This application is the U.S. National Stage of International Application PCT/US2019/035512, filed Jun. 5, 2019, and claims priority to U.S. provisional application 62/681,342, filed Jun. 6, 2018, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Sep. 10, 2019, is named 105894-0113_SL.txt and is 162,737 bytes in size.

### FIELD

The present disclosure relates generally to methods of increasing or improving circulation and the treatment of cardiovascular disease, including Buerger's Disease, critical limb ischemia, peripheral artery disease, thrombus formation, and atherosclerotic disease in the heart, brain and other organs, by administering an anti-nicotine agent such as nicotine-binding antibodies and/or nicotine-degrading enzymes to tobacco smokers or persons who ingest nicotine (such as from e-cigarettes, chewing tobacco, nicotine replacement therapy, etc.). In accordance with the disclosure, the disclosed anti-nicotine agents treat cardiovascular diseases in persons with nicotine present in their blood in addition to facilitating smoking cessation and/or facilitating and maintaining abstinence.

### BACKGROUND

The following discussion is merely provided to aid the reader in understanding the disclosure and is not admitted to describe or constitute prior art thereto.

Nicotine is a bitter-tasting, parasympathomimetic alkaloid compound that naturally occurs in large amounts in the leaves of tobacco plants. Nicotine is a nicotinic acetylcholine receptor (nAChR) agonist and functions physiologically as a stimulant. Nicotine is both addictive and toxic, and its ingestion or inhalation (or other mode of administration) have been associated with cardiovascular disease, potential birth defects, and poisoning (in overdose).

The most common source of body nicotine is tobacco smoking. Smoking is a global healthcare problem, largely due to the addictiveness of nicotine. The World Health Organization estimates that there are 1.1 billion smokers worldwide today and nearly six million tobacco-related deaths each year. If current smoking patterns continue, smoking will cause some 8 million deaths each year by 2030. According to the U.S. Center for Disease Control (CDC), tobacco use is the single leading preventable cause of death in the U.S., responsible for over 480,000 deaths each year. In the U.S., direct health care costs due to treating smoking and smoking-related illness are estimated to exceed $170 billion per year and $156 billion in lost productivity. The CDC estimates that, among the 36 million adult smokers in the U.S., 70% want to quit, but less than five percent of those who try to quit remain smoke-free after 12 months. In addition, 16 million individuals in the U.S. have a smoking related illness and yet 40% continue to smoke—a significant proportion of these individuals have cardiovascular disease.

Of the numerous health consequences associated with smoking, tobacco use, and other forms of nicotine consumption, nicotine-related cardiovascular diseases are among the most serious. For example, smoking and tobacco consumption have been linked to the development of Buerger's Disease (i.e., thromboangiitis obliterans or TAO), critical limb ischemia, peripheral artery disease, atherosclerotic disease, and vascular inflammation or blockage in the heart, brain and other organs. Indeed, smoking and tobacco consumption are linked to numerous serious, life-threatening conditions with few treatment options.

Buerger's Disease is a segmental inflammatory occlusive disorder that affects small- and medium-sized arteries, and arm and leg veins. It is exclusively associated with smoking, and it can occur in young smokers as well as old. Clinically, it manifests with signs of arterial insufficiency in the extremities and impaired vasodilation. The associated thromboses are often occlusive and sometimes display moderate, nonspecific inflammatory infiltrate, consisting mostly of polymorphonuclear leukocytes, mononuclear cells and rare multinuclear giant cells. The immune system's response to nicotine appears to play a critical role in the etiology of Buerger's Disease, but knowledge about immunological aspects involved in the progression of vascular tissue inflammation, and consequently, the evolution of this disease, is still limited. Some groups have suggested that Buerger's Disease may be an autoimmune disorder initiated by an unidentified antigen in the vascular endothelium, possibly nicotine or a nicotine metabolite. Clinically, the only established treatment to stop the progression of Buerger's Disease is smoking cessation. There are no FDA approved drugs or devices for treatment of Buerger's Disease. However, the clinical literature has reported partial success in reducing symptoms of Buerger's Disease by sympathectomy or use of the vasodilators cilostazol, and prostaglandin analogues (prostacyclin or prostaglandin E).

Critical limb ischemia (CLI) is a serious condition in which there is inadequate blood flow and oxygen to the limbs, hands/fingers or feet/toes. It is generally caused by a narrowing or blockage of the small arteries in the limbs. CLI causes tissue damage, pain, poorly healing ulceration, gangrene, and loss of digits or other portion of limbs due to amputation. Like Buerger's Disease, this condition will not improve on its own without treatment.

Peripheral artery disease (PAD) is a disease in which plaques made up of fat, cholesterol, calcium, fibrous tissue, and other substances builds up in the arteries that carry blood to the head, organs, and limbs. Over time, plaque can harden and narrow the arteries. This limits the flow of oxygen-rich blood to the organs and other parts of the body. PAD usually affects the arteries in the legs, but it also can affect the arteries that carry blood from the heart to the head, arms, kidneys, and stomach. The primary risk factor for developing PAD is smoking. Cilostazol or pentoxifylline can improve symptoms in some, but there is no strong evidence to suggest that it improves the quality of life, decreases mortality, or decreases the risk of cardiovascular events associated with smoking/nicotine exposure. Revascularization surgeries like angioplasty and vascular bypass are considered to be the most efficacious options for patients with PAD.

Atherosclerotic disease (i.e., atherosclerosis) refers to a buildup of fats, cholesterols, and other substances in and on the walls of the arteries. These buildups, also known as plaques, cause the arteries to become thick and stiff and can impact blood flow to any effected organ, such as the heart, brain, or other organs. When atherosclerosis affects the heart, it can cause chest pain and pressure, and when it affects the brain it may cause sudden numbness or weakness, difficulty speaking/slurred speech, temporary loss of vision, or a drooping face—all of which may also signal a transient ischemic attack. The arms and legs are also commonly affected by atherosclerosis, which may lead to pain when walking or moving, and the kidneys are commonly impacted as well. While there are numerous medicinal (e.g., cholesterol medications, anti-platelet medications, beta blockers, ACE inhibitors, etc.) and surgical (e.g., angioplasty, stent placement, bypass surgery) interventions available for the treatment of atherosclerosis, the primary recommended modification for tobacco users is to cease or abstain from tobacco use, which many users find difficult to achieve.

Thus, there remains a need for effective agents for treating cardiovascular diseases associated with smoking and nicotine consumption, such as Buerger's Disease, critical limb ischemia, peripheral artery disease, vascular inflammation, thrombus formation, and atherosclerotic disease. The present disclosure fulfills this need.

### SUMMARY

Described herein are methods of treating nicotine users (e.g., smokers and subjects using other forms of nicotine ingestion) with cardiovascular disease, such as one or more of Buerger's Disease, critical limb ischemia, peripheral artery disease, thrombus formation, and atherosclerotic disease (e.g., atherosclerosis of arteries in the heart, brain and other organs), by administering an anti-nicotine agent such as nicotine-binding antibodies and/or nicotine-degrading enzymes.

In one aspect, the present disclosure provides methods of improving circulation or treating a cardiovascular disease in a subject in need thereof, comprising administering to the subject a therapeutically effective amount of an anti-nicotine agent.

In some embodiments, the anti-nicotine agent may be a nicotine-binding antibody or a nicotine-binding fragment thereof, while in some embodiments, the anti-nicotine agent may be a nicotine-degrading enzyme.

In some embodiments, the anti-nicotine agent may be administered by a route of administration selected from intravenously, subcutaneously, intramuscularly, and intraperitoneally.

In some embodiments, the subject being treated is a current consumer of a nicotine product. For example, in some embodiments, the subject may be an active smoker, e-cigarette user, or tobacco user. In some embodiments, the subject may be undergoing smoking cessation therapy, while in some embodiments, the subject may be abstaining from one or more or all of smoking, e-cigarette use, or tobacco use.

In some embodiments, the cardiovascular disease may be selected from one or more of Buerger's Disease, critical limb ischemia, peripheral artery disease, thrombus formation, and atherosclerotic disease (e.g., atherosclerosis affecting the heart or brain). In some embodiments, the method may be effective to reduce one or more symptoms of cardiovascular disease in the subject selected from blood pressure, vasoconstriction, and heart rate.

In another aspect, the present disclosure provides anti-nicotine agents for use in treating cardiovascular disease and/or improving circulation or diminishing further impairment of circulation in a subject in need thereof. In some embodiments, the anti-nicotine agent may be a nicotine-binding antibody (or nicotine-binding fragment thereof). In other embodiments, the anti-nicotine agent may be a nicotine-degrading enzyme. In any embodiments, the cardiovascular disease may be selected from one or more of Buerger's Disease, critical limb ischemia, peripheral artery disease, thrombus formation, and atherosclerotic disease. In any embodiments, the anti-nicotine agent may be formulated for administration by a route selected from intravenously, subcutaneously, intramuscularly, and intraperitoneally. In any embodiments, the subject may be an active smoker, e-cigarette user, and/or tobacco user. In some embodiments, the subject is undergoing smoking cessation therapy. In some embodiments, the subject is abstaining from one or more or all of smoking, e-cigarette use, and/or tobacco use.

In another aspect, the present disclosure provides uses of an anti-nicotine agent in the preparation of a medicament for treating cardiovascular disease and/or improving circulation in a subject in need thereof. In some embodiments, the anti-nicotine agent may a nicotine-binding antibody (or nicotine-binding fragment thereof) or a nicotine-degrading enzyme (or nicotine-degrading fragment thereof). In some embodiments, the cardiovascular disease may be selected from one or more of Buerger's Disease, critical limb ischemia, peripheral artery disease, thrombus formation, and atherosclerotic disease. In any embodiments, the subject may be an active smoker, e-cigarette user, and/or tobacco user. In some embodiments, the subject is undergoing smoking cessation therapy. In some embodiments, the subject is abstaining from one or more or all of smoking, e-cigarette use, and/or tobacco use.

The foregoing general description and following detailed description are exemplary and explanatory and are intended to provide further explanation of the invention.

### BRIEF DESCRIPTION OF DRAWINGS

FIG. 1 shows brain and serum nicotine levels after single nicotine exposure and pretreatment with 8D1-IgG4 (Mean±SD; p<0.0001 by one-way ANOVA with Dunnett's test for individual comparisons). The disclosed anti-nicotine agents sequester nicotine in serum and prevent it from entering into, for example, the brain, where it can produce physiological effects. The top panel shows increased serum concentrations of nicotine and the bottom panel shows decreased concentrations of brain nicotine with increasing doses of an anti-nicotine antibody.

FIG. 2 shows brain and nicotine levels following repeated nicotine exposure after pretreatment with 8D1-IgG4 (Mean±SD; p<0.0001 by one-way ANOVA with Dunnett's test for individual comparisons). Nicotine concentration in the brain is maintained at low levels by anti-nicotine antibodies even after repeated administration of nicotine which simulates very heavy smoking.

FIG. 3 shows reduction of blood and brain nicotine concentrations by NicA2. Rats were pretreated with NicA2 i.v. and 5 min later received nicotine 0.03 mg/kg i.v. Groups of rats had nicotine levels measured at 1, 3 or 5 min. Blood (upper panel) and brain (lower panel) nicotine concentrations were reduced by NicA2 in a dose- and time-related manner, with substantial NicA2 effects at doses of ≥5 mg/kg, and with greater reduction of nicotine concentrations at 3 and 5 min than at 1 min. **p<0.01, ***p<0.001 compared to BSA using Bonferroni-corrected Welch's t tests. Mean±SD, n=8/group.

FIG. 4 shows the effects of NicA2 in rats receiving multiple nicotine doses. After pretreatment with 10 mg/kg NicA2, rats received either one nicotine dose of 0.03 mg/kg i.v. or 5 nicotine doses at 10 min intervals. Numbers above bars are the percent reduction of nicotine concentrations 3 min after the nicotine dose compared to BSA control, in blood (upper panel) and brain (lower panel). *** p<0.001, two-tailed unpaired t tests with Welch's correction. Mean±SD, n=10/group.

FIG. 5 shows change in mean arterial pressure before and after nicotine challenge in rats pretreated with 8D1-IgG4. Anti-nicotine antibodies prevent a large increase in mean arterial pressure (MAP) induced by nicotine, when administered prior to nicotine exposure. Based on these results, a significant dose-dependent attenuation of nicotine-induced increases in MAP by 8D1-IgG4 was observed (p=0.033; non-parametric one-way ANOVA using Friedman's test and Dunn's correction for multiple comparisons.). The top panel shows the average change in MAP across several days at varying doses, and the bottom panel shows the strong correlation between the plasma antibody concentration and the change in MAP.

FIG. 6 shows average change in mean arterial pressure before and after each repeated nicotine dose in rats pretreated with 8D1-IgG4. Pretreatment with anti-nicotine antibodies prevents large increases in MAP even after repeated administration of nicotine.

### DETAILED DESCRIPTION

Described herein are methods of treating cardiovascular disease by administering an anti-nicotine agent such as nicotine-binding antibodies and/or nicotine-degrading enzymes.

### I. Definitions

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the phrases “therapeutically effective amount” and “therapeutic level” mean that drug dosage or plasma concentration in a subject that provides the specific pharmacological effect for which the drug is administered in a subject that is a current users of nicotine and in need of such treatment, i.e., to reduce, ameliorate, or eliminate the symptoms or effects of cardiovascular diseases such as Buerger's Disease, critical limb ischemia, peripheral artery disease, thrombus formation, and atherosclerotic disease. It is emphasized that a therapeutically effective amount or therapeutic level of a drug will not always be effective in treating the conditions described herein, even though such dosage is deemed to be a therapeutically effective amount by those of skill in the art. For convenience only, exemplary dosages, drug delivery amounts, therapeutically effective amounts, and therapeutic levels are provided below. The therapeutically effective amount may vary based on the route of administration and dosage form, the age and weight of the subject, and/or the subject's condition, including the severity of the cardiovascular disease and the location or organ involvement of the pathophysiology.

The terms “treatment” or “treating” as used herein with reference to cardiovascular diseases means reducing, ameliorating or eliminating one or more symptoms or effects of the cardiovascular disease, such as Buerger's Disease, critical limb ischemia, peripheral artery disease, thrombus formation, and atherosclerotic disease in current smokers or other individuals who have nicotine present in their body. Symptoms of Buerger's Disease, critical limb ischemia, peripheral artery disease, thrombus formation, and atherosclerotic disease include, but are not limited to, pain, impaired circulation, ulcerations, and gangrene. Buerger's Disease, critical limb ischemia, peripheral artery disease, thrombus formation, and atherosclerotic disease may lead to complications that result in the need for amputation of an extremity, and the present treatments decrease the risk of needing an amputation.

The terms “abstinence” or “abstain” as used herein with reference to smoking, tobacco use, or nicotine use refers to an individual not smoking, using tobacco, or using nicotine for any period of time less than 6 months. Periods of abstinence need not be continuous and may be interspersed with periods of smoking, tobacco use, or nicotine use. Accordingly, an individual temporarily abstaining from smoking may still be a chronic smoker or tobacco user.

The terms “cessation” or “cease” as used herein with reference to smoking, tobacco use, or nicotine use refers to an individual continuously abstaining from smoking, tobacco use, or nicotine use for at least 6 months. An individual may undergo therapy in order to cease smoking (i.e., smoking cessation therapy), which may include but is not limited to cognitive or behavioral therapy, nicotine replacement therapy (e.g., gums or patches), treatment with a nicotine-binding antibody or nicotine degrading enzyme, or treatment with medications like varenicline tartrate or bupropion HCl.

The term “reduction” with respect to smoking or other tobacco/nicotine use means a decrease in the amount of smoking or other tobacco/nicotine use by at least about 25%, at least about 50%, at least about 75%, at least about 80%, at least about 85%, at least about 90%, or at least about 95%. An individual may experience therapeutic benefits from a reduction in smoking or tobacco/nicotine use. Depending on the degree of reduction in smoking or other tobacco/nicotine use, the benefits of reduction may last for at least about one month or more.

The terms “individual,” “subject,” and “patient” are used interchangeably herein, and refer to any individual mammal subject, e.g., bovine, canine, feline, equine, or human. For the purposes of the present disclosure, an “individual,” “subject,” or “patient” may be presently using, consuming, ingesting, or be otherwise exposed to nicotine in some form (e.g., smoking, using e-cigarettes, using smokeless tobacco, exposed to second-hand smoke, etc.) during the course of treatment according to the disclosed methods. For example, the subject may be a nicotine user, undergoing a smoking cessation program, and/or abstaining from nicotine use.

### II. Nicotine, Addiction, and Toxicity

Nicotine is a nitrogen-containing chemical made by several types of plants including tobacco and other members of the nightshade family. When humans, mammals and most other types of animals are exposed to nicotine, it increases their heart rate, heart muscle oxygen consumption rate, and heart stroke volume. The consumption of nicotine is also linked to raised alertness, euphoria, and a sensation of being relaxed. However, nicotine is highly addictive. The American Heart Association says that nicotine (from smoking tobacco) is one of the hardest substances to quit, at least as hard as heroin.

The structure of nicotine is shown in Formula I below.

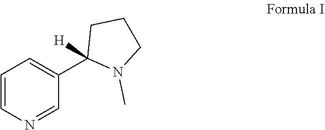

Nicotine is an agonist for α4β2 nicotine cholinergic receptor (nAChR) subtype in the central nervous system (CNS). This neuronal α4β2 nAChR is thought to mediate nicotine dependence. Stimulation of central nAChRs by nicotine releases dopamine, which is critical to the reinforcing effects of nicotine. nAChR desensitization and subsequent upregulation after chronic nicotine exposure is thought to play roles in both tolerance and dependence, with unoccupied neuronal α4⊖2 nAChRs promoting craving and withdrawal symptoms.

### III. Nicotine's Detrimental Effects on the Cardiovascular System

Nicotine's effects on the vasculature are detrimental to patients with cardiovascular disease, where additional peripheral vessel narrowing due to nicotine-induced vasoconstriction or thrombus formation can trigger or worsen symptoms. S-(−)-nicotine, the nicotine stereoisomer found in tobacco (>99%), activates the α3β4 nicotinic cholinergic receptor subtype on peripheral postganglionic sympathetic nerve endings, autonomic ganglia and chromaffin cells of the adrenal gland causing release of catecholamines (i.e., epinephrine and norepinephrine) into the bloodstream. Circulating catecholamines activate α-adrenoreceptors located on vascular smooth muscle, constricting blood vessels. Catecholamine-induced α-adrenoreceptor stimulation increases total vascular peripheral resistance.

In addition to its effects on vasoconstriction, nicotine prevents compensatory vasorelaxation. Nicotine has inhibitory effects on vessel vasorelaxation in the periphery, presumably through reactive oxygen species generation. It is believed that the production of oxygen free radicals by nicotine impairs nitric oxide synthase-dependent vasoreactivity. Furthermore, a number of nAChRs are present in the endothelial cells that line the internal surfaces of blood vessels. Activation of α7-nAChRs by nicotine can induce endothelium-dependent relaxation. However, since all nAChR subtypes exhibit desensitization after prolonged exposure to agonists, nAChR mediated endothelium-dependent vasorelaxation may be inhibited in cardiovascular disease patients who are heavy tobacco users. This cumulative inhibition of vasorelaxation by nicotine is particularly important in patients with Buerger's Disease since they have impaired nitroglycerin-induced vasodilation when compared with that for healthy controls.

The combination of effects of nicotine-induced vasoconstriction and impaired vasodilation has implications in the healing of ischemic ulcers. Ischemic skin ulcers form in Buerger's Disease and Critical Limb Ischemia patients as a result of impaired blood flow to the extremities and subsequent necrosis. Nicotine has direct local vasoconstrictive effects (in addition to its central action in triggering catecholamine release from the adrenal gland) which may be part of the underlying pathogenesis of skin ischemic disease associated with cigarette smoking or other tobacco products.

Endothelial activation occurs after tobacco-induced injury, and nicotine stimulates tissue factor expression in endothelial and smooth muscle cells, shifting them towards a pro-thrombotic state. High dose intravenous nicotine administration increased arteriolar thrombosis with an increased endothelial P-selectin expression, important for the initial attachment of leukocytes to the vessel wall during thrombus formation. Nicotine also increases platelet adhesiveness, and increases the risk for microvascular occlusion and subsequent exacerbation of symptoms.

While controversy exists regarding whether nicotine induces or inhibits inflammation, many studies suggest nicotine is pro-inflammatory. The diagnostic acute-phase lesion of Buerger's Disease involves inflammation of all layers of the vessel wall and is associated with an inflammatory thrombus consisting mostly of polymorphonuclear leukocytes, mononuclear cells, and occasional multinucleated giant cells. Chronic nicotine exposure enhances proinflammatory tumor necrosis factor a (TNF-α) secretion by endothelial cells. Increased levels of TNF-α have been found in the plasma of Buerger's Disease patients and endothelial cells of these patients showed morphological signs of activation indicating that vascular lesions are associated with TNF-α. In addition, nicotine at levels comparable to smoker's plasma is chemotactic for human neutrophils, further implicating it in inflammatory thrombus formation.

Normal functioning of arterial vessels require substantial flexibility (distendability) of the vessels to expand and contract their volume in response to the normal pumping of the heart which generates waves of increasing and decreasing blood pressure. Distendability is impaired when endothelial cells lining the interior walls of arteries undergo inflammation due to nicotine or to cigarette smoke induced inflammatory substances, such as C-reactive protein, interleukin-6 (IL-6) or other inflammatory cytokines or macrophages (Libby, 2007; McEvoy 2015; Barua 2015; Liu 2017). Also, over time arterial inflammation is also associated with the development of chronic arterial atherosclerosis, which progresses clinically to chronic peripheral arterial disease (PAD). If smoking is maintained in PAD subjects, continuation of the inflammation/atherogenic process worsens the distendability of blood vessels, often leading to critical limb ischemia (CLI) and ultimately to death due to coronary artery disease. There are no broad spectrum anti-inflammatory agents available which inhibit nicotine or cigarette smoke induced vascular inflammation. Therefore, the best medical approach is to diminish or stop the exposure of the vasculature to the inflammatory agents of nicotine or cigarette smoke.

### IV. Cardiovascular Diseases

Certain cardiovascular diseases are associated with the consumption or use of nicotine and nicotine-containing products such as cigarettes, e-cigarettes, smokeless tobacco and other tobacco products. For the purposes of the present disclosure, “cardiovascular diseases” refers to cardiovascular diseases that are associated with the use or consumption of nicotine and/or nicotine-containing products. These types of cardiovascular disease include Buerger's Disease, critical limb ischemia (CLI), peripheral artery disease (PAD), atherosclerotic disease (e.g., atherosclerosis of the heart or brain).

Buerger's Disease is a particular type of peripheral arterial disease that is strongly linked to smoking or tobacco exposure in virtually all subjects diagnosed with Buerger's Disease. If the tobacco exposure persists for years, severe pain occurs due to compromised blood flow to the upper and lower limbs. Depending on disease severity and amount of smoking, patients may develop gangrene with the consequent need for amputation of fingers, toes or more extensive portions of the limbs. It is widely recognized that smoking cessation or ending exposure to smokeless tobacco or other forms of nicotine is the only way to stop or slow disease progression. Otherwise, there is no accepted pharmacological treatment of the underlying disorder, although IV infusion of a prostaglandin (potent vasodilator) may be beneficial. Blood vessel transplants to affected areas may also provide temporary relief.

The incidence of Buerger's disease in the U.S. is about 40,000-70,000 patients, and the disorder is considered an orphan disease. A greater incidence exists in the Middle East, Asia, and parts of Europe. Unfortunately, many subjects with Buerger's Disease are unable to quit smoking (as often occurs in patients with COPD or with lung cancer, for whom smoking is the likely primary cause).

Given the correlation between urinary cotinine levels and worsening symptom exacerbations among active smokers with Buerger's Disease, it is critical for patients with Buerger's Disease to avoid nicotine and tobacco products or to be treated by an agent that blocks nicotine's access to the peripheral vasculature. Nicotine's ability to induce vasoconstriction, inhibition of vasorelaxation, impairment of skin ulcer healing, and thrombus formation are part of the underlying pathophysiology of the Buerger's Disease and therefore an anti-nicotine agent may be beneficial in treating Buerger's Disease patients who have not been able to stop smoking or stop use of smokeless tobacco or other forms of nicotine intake (e-cigarettes for example). The present approach may also be beneficial in patients receiving nicotine replacement therapy (NRT), as this type of cessation therapy still results in circulating nicotine. Importantly, the disclosed anti-nicotine agents bind and/or degrades nicotine in the periphery, thereby preventing activation of nAChRs that mediate vasoconstriction. Reduction of active nicotine in the periphery will also prevent free radical formation and prevent desensitization of nAChRs on endothelial cells that modulate vasodilation.

PAD is an umbrella term that refers to a general vascular condition involving the narrowing and reduced blood flow in the arteries, which may cause or enhance the pathology of related indications such as Reynaud's disease, CLI, and atherosclerosis.

While it is clear that smoking cessation or abstinence from tobacco would be beneficial to patients suffering from or at risk of developing these cardiovascular diseases, many are unable to quit due to the addictiveness of nicotine. The methods disclosed herein are based on the surprising finding that administration of an anti-nicotine agent as described herein can treat cardiovascular diseases such as Buerger's Disease, CLI, PAD, thrombus formation, and atherosclerotic disease, among other diseases (e.g., Reynaud's disease), even when the subject is unable or unwilling to cease consumption of nicotine/tobacco. Without being bound by theory, the methods described herein may be effective by sequestering or degrading nicotine and thereby preventing nicotine from binding a cognate receptor and/or inducing a physiological response (e.g., vasoconstriction) or an immune response (which may be particularly relevant in the context of Buerger's Disease).

In specific embodiments, the methods described herein may be effective to reduce, ameliorate, or eliminate symptoms or effects of a cardiovascular disease such as high blood pressure induced by nicotine (e.g., an increase of at least about 5, 10, 15, 20, 25, 30, 35, 40, or 45 or more mm Hg systolic or 5, 10, 15, 20, 25, 30, 35, 40, or 45 or more mm Hg diastolic above the normal, resting blood pressure in the absence of nicotine), increased mean arterial pressure (MAP) induced by nicotine, elevated heart rate induced by nicotine, and vasoconstriction induced by nicotine. In further specific embodiments, the methods described herein may be effective to reduce, ameliorate, or eliminate symptoms or effects of a cardiovascular disease such as acute or chronic inflammation and/or thrombosis of arteries and/or veins, including arteries and/or veins of the hands and/or feet.

### V. Other Diseases and Conditions Suitable for Treatment

Beyond the cardiovascular indications discussed above, the disclosed compositions and methods may be suitable for treatment of a variety of diseases and conditions associated with smoking and/or the use and consumption of nicotine. For example, in addition to the cardiovascular disorders induced by or exacerbated by nicotine, described herein, nicotine exposure is well established to cause or worsen the functioning of many body systems upon which good health depends. Addiction to nicotine drives individuals to continue to smoke and use other forms of nicotine long-term, making it difficult to treat many of the underlying pathologies associated with nicotine inhalation, consumption, ingestion, or exposure. Further, the smoke directly irritates and impairs functioning of the respiratory system, apart from its carcinogenic effects.

The following are additional diseases/disorders that are either caused or worsened by nicotine: cataracts, blindness (e.g., macular degeneration), excessive tearing, stinging of the eyes, stroke, nicotine addiction, cancer (e.g., cancer of the nasal cavity, paranasal sinus, lungs, lips, mouth, throat, larynx, pharynx, tracheal, esophageal, gastric, colon, pancreatic, breast, liver, prostate, bladder, kidney, ureter, cervical, ovarian, bone marrow, acute myeloid leukemia, etc.), chronic rhinosinusitis, impaired sense of smell, periodontal disease, dental decay, impaired sense of taste, hearing loss, ear infection, acute or chronic bronchitis, chronic obstructive pulmonary disease (COPD), emphysema, worsening of respiratory infections (tuberculosis, pneumonia, influenza), worsening of asthma, chronic cough, shortness of breath, excess sputum production, abdominal aortic aneurysm, peptic ulcer (esophagus, stomach, upper GI tract), reduced fertility, impotence, premature ovarian failure, early menopause, painful menstruation, Reynaud's disease, poor circulation, wrinkling, premature aging, loss of skin tone, osteoporosis, bone fracture (e.g., hip, knee and spinal fracture), rheumatoid arthritis, back problems; impaired wound healing, poor post-surgical recovery, leg pain, cold feet, gangrene, deep vein thrombosis, impaired resistance to infection, increased risk of allergic disorders, increased risk of diabetes, diabetic nephropathy, diabetic skin ulcers diabetic circulatory disorders, neuropathy, diabetes retinal disorders, diabetes related ulceration, vasculitis, amputation, and sudden death.

Of these disease/conditions, the disclosed methods are particularly useful for improving wound healing and surgical recovery, as well as treating diabetes and diabetic complications as a result of the vasodilating effects of the disclosed anti-nicotine agents in nicotine users. Administration of the disclosed anti-nicotine agents degrades or sequesters nicotine in circulation, thereby preventing vasoconstriction and improving circulation and blood flow in individuals with nicotine in their system (e.g., smokers). Increasing blood flow to a wound or surgical cite will promote healing and improve patient outcomes, and similar benefits would be garnered in diabetic patients with circulation problems in their extremities.

### VI. Nicotine-Degrading Enzymes

In one aspect, the anti-nicotine agent used in the disclosed methods is a nicotine-degrading enzyme. Examples of nicotine-degrading enzymes include the wild-type NicA2 enzyme SEQ ID NO: 1, and variants thereof that degrade nicotine, examples of which are set forth in Table 1 below. In some embodiments, a variant has at least one substitution, addition, or deletion relative to SEQ ID NO: 1 that increases the nicotine-degrading activity and/or decreases the immunogenicity of the variant relative to the wild-type enzyme.

NicA2 (nicotine oxidoreductase; PPS 4081; GenBank accession number: AEJ14620.1), was isolated from Pseudomonas putida strain S16. See, e.g., Tang et al., PLOS GENETICS, 9(10): e1003923 (2013). The activity of NicA2 is the first committed step of S16+s degradation of nicotine, catalyzing the oxidation of nicotine to N-methylmyosmine. It is reported to be an essential enzyme in the P. putida S16 metabolic cascade responsible for breaking down nicotine. A structural analysis of the wild-type NicA2 enzyme has been reported in Tararina et al., Biochem. 55:6595-98 (2016).

As noted above, in some embodiments, a variant of the wild-type NicA2 enzyme with improved activity and/or decreased immunogenicity is used. In some embodiments, the variants may have an amino acid identity that is about 80, about 85, about 90, about 95, about 96, about 97, about 98, or about 99 percent of wild-type NicA2. In some embodiments, the disclosed variants may share about 80, about 85, about 90, about 95, about 96, about 97, about 98, or about 99 percent homology with wild-type NicA2. The amino acid sequence of wild-type NicA2, and exemplary variants thereof are set forth in Table 1 below. The disclosed variants were produced with a linker and His-tag (GGGGSGSGHHHHHH, SEQ ID NO: 51) at the C-terminal end, which was subsequently removed. The His-tag was used to assist in purification of the variants, but other means or methods of purification that do not require a His-tag may also be used.

TABLE 1
Amino Acid Sequences of NicA2 and Exemplary Variants

| | SEQ | |
|---|---|---|
| | ID | |
| Enzyme | NO. | Sequence |
| Wild-Type | 1 | M*SDKTKTNEGFSRRSF [symbol] GVAGLGAIDAASATQKT |
| NicA2 | | NRASTVKGGFDYDVVVVGGGFAGATAARECG [symbol] E |
| | | ARSRLGGRTFTSRFAGQEIEFGGAWVHWLQPHVWAEMQRYG |
| | | LGVVEDPLTNLDKTLIMYNDGSVESISPDEFGKNIRIAFEKLCH |
| | | DAWEVFPRPREPMFTERARELDKSSVLDRIKTLGLSRLQQAQIN |
| | | SYMALYAGETTDKFGLPGVLKLFACGGWNYDAFMDTETHYRI |
| | | QGGT [symbol] DSGAEVRMSVPVTAVEQVNGGVKIKTDDD |
| | | EIITAGV [symbol] TYKHIGFTPALSKGKQRFIKEGQLSKGAK |
| | | LYVHVKQNLGRVFAFADEQQPLNWVQTHDYSDE [symbol] A |
| | | RKETIDVNDRDAVTREVQKMFPGVEVLGTAAYDWTADPFSLG |
| | | AWAAYGVGQLSRLKDLQAAEGR [symbol] NGWHANIDGA |
| | | VESGLRAGREVKQLLS**† |
| NicA2Δ50 | 2 | GFDYDVVVVGGGFAGATAARECGLQGYRTLLLEARSRLGGRT |
| (N-terminal | | FTSRFAGQEIEFGGAWVHWLQPHVWAEMQRYGLGVVEDPLT |
| deletion of | | NLDKTLIMYNDGSVESISPDEFGKNIRIAFEKLCHDAWEVFPRP |
| residues 1-50) | | HEPMFTERARELDKSSVLDRIKTLGLSRLQQAQINSYMALYAG |
| | | ETTDKFGLPGVLKLFACGGWNYDAFMDTETHYRIQGGTIGLIN |
| | | AMLTDSGAEVRMSVPVTAVEQVNGGVKIKTDDDEIITAGVVV |
| | | MTVPLNTYKHIGFTPALSKGKQRFIKEGQLSKGAKLYVHVKQN |
| | | LGRVFAFADEQQPLNWVQTHDYSDELGTILSITIARKETIDVND |
| | | RDAVTREVQKMFPGVEVLGTAAYDWTADPFSLGAWAAYGVG |
| | | QLSRLKDLQAAEGRILFAGAETSNGWHANIDGAVESGLRAGRE |
| | | VKQLLS |
| NicA2Δ25 | 3 | GVAGLGAIDAASATQKTNRASTVKGGFDYDVVVVGGGFAGA |
| (N-terminal | | TAARECGLQGYRTLLLEARSRLGGRTFTSRFAGQEIEFGGAWV |
| deletion of | | HWLQPHVWAEMQRYGLGVVEDPLTNLDKTLIMYNDGSVESIS |
| residues 1-25) | | PDEFGKNIRIAFEKLCHDAWEVFPRPHEPMFTERARELDKSSVL |
| | | DRIKTLGLSRLQQAQINSYMALYAGETTDKFGLPGVLKLFACG |
| | | GWNYDAFMDTETHYRIQGGTIGLINAMLTDSGAEVRMSVPVT |
| | | AVEQVNGGVKIKTDDDEIITAGVVVMTVPLNTYKHIGFTPALS |
| | | KGKQRFIKEGQLSKGAKLYVHVKQNLGRVFAFADEQQPLNWV |
| | | QTHDYSDELGTILSITIARKETIDVNDRDAVTREVQKMFPGVEV |
| | | LGTAAYDWTADPFSLGAWAAYGVGQLSRLKDLQAAEGRILFA |
| | | GAETSNGWHANIDGAVESGLRAGREVKQLLS |
| NicA2Δ38 | 4 | TQKTNRASTVKGGFDYDVVVVGGGFAGATAARECGLQGYRT |
| (N-terminal | | LLLEARSRLGGRTFTSRFAGQEIEFGGAWVHWLQPHVWAEMQ |
| deletion of | | RYGLGVVEDPLTNLDKTLIMYNDGSVESISPDEFGKNIRIAFEK |
| residues 1-38) | | LCHDAWEVFPRPHEPMFTERARELDKSSVLDRIKTLGLSRLQQ |
| | | AQINSYMALYAGETTDKFGLPGVLKLFACGGWNYDAFMDTET |
| | | HYRIQGGTIGLINAMLTDSGAEVRMSVPVTAVEQVNGGVKIKT |
| | | DDDEIITAGVVVMTVPLNTYKHIGFTPALSKGKQRFIKEGQLSK |
| | | GAKLYVHVKQNLGRVFAFADEQQPLNWVQTHDYSDELGTILS |
| | | ITIARKETIDVNDRDAVTREVQKMFPGVEVLGTAAYDWTADPF |
| | | SLGAWAAYGVGQLSRLKDLQAAEGRILFAGAETSNGWHANID |
| | | GAVESGLRAGREVKQLLS |
| NicA2 | 5 | SDKTKTNEGFSRRSFIGSAAVVTAGVAGLGAIDAASATQKTNR |
| A107R | | ASTVKGGFDYDVVVVGGGFAGATAARECGLQGYRTLLLEARS |
| (A107R | | RLGGRTFTSRFAGQEIEFGGRWVHWLQPHVWAEMQRYGLGV |
| substitution) | | VEDPLTNLDKTLEVIYNDGSVESISPDEFGKNIRIAFEKLCHDAW |
| | | EVFPRPHEPMFTERARELDKSSVLDRIKTLGLSRLQQAQINSYM |
| | | ALYAGETTDKFGLPGVLKLFACGGWNYDAFMDTETHYRIQGG |
| | | TIGLINAMLTDSGAEVRMSVPVTAVEQVNGGVKIKTDDDEIITA |
| | | GVVVMTVPLNTYKHIGFTPALSKGKQRFIKEGQLSKGAKLYVH |
| | | VKQNLGRVFAFADEQQPLNWVQTHDYSDELGTILSITIARKETI |
| | | DVNDRDAVTREVQKMFPGVEVLGTAAYDWTADPFSLGAWAA |
| | | YGVGQLSRLKDLQAAEGRILFAGAETSNGWHANIDGAVESGL |
| | | RAGREVKQLLS |
| NicA2A107K | 6 | SDKTKTNEGFSRRSFIGSAAVVTAGVAGLGAIDAASATQKTNR |
| (A107K | | ASTVKGGFDYDVVVVGGGFAGATAARECGLQGYRTLLLEARS |
| substitution) | | RLGGRTFTSRFAGQEIEFGGKWVHWLQPHVWAEMQRYGLGV |
| | | VEDPLTNLDKTLIMYNDGSVESISPDEFGKNIRIAFEKLCHDAW |
| | | EVFPRPHEPMFTERARELDKSSVLDRIKTLGLSRLQQAQINSYM |
| | | ALYAGETTDKFGLPGVLKLFACGGWNYDAFMDTETHYRIQGG |
| | | TIGLINAMLTDSGAEVRMSVPVTAVEQVNGGVKIKTDDDEIITA |
| | | GVVVMTVPLNTYKHIGFTPALSKGKQRFIKEGQLSKGAKLYVH |
| | | VKQNLGRVFAFADEQQPLNWVQTHDYSDELGTILSITIARKETI |
| | | DVNDRDAVTREVQKMFPGVEVLGTAAYDWTADPFSLGAWAA |
| | | YGVGQLSRLKDLQAAEGRILFAGAETSNGWHANIDGAVESGL |
| | | RAGREVKQLLS |
| NicA2A107T | 7 | SDKTKTNEGFSRRSFIGSAAVVTAGVAGLGAIDAASATQKTNR |
| (A107T | | ASTVKGGFDYDVVVVGGGFAGATAARECGLQGYRTLLLEARS |
| substitution) | | RLGGRTFTSRFAGQEIEFGGTWVHWLQPHVWAEMQRYGLGV |
| | | VEDPLTNLDKTLIMYNDGSVESISPDEFGKNIRIAFEKLCHDAW |
| | | EVFPRPHEPMFTERARELDKSSVLDRIKTLGLSRLQQAQINSYM |
| | | ALYAGETTDKFGLPGVLKLFACGGWNYDAFMDTETHYRIQGG |
| | | TIGLINAMLTDSGAEVRMSVPVTAVEQVNGGVKIKTDDDEIITA |
| | | GVVVMTVPLNTYKHIGFTPALSKGKQRFIKEGQLSKGAKLYVH |
| | | VKQNLGRVFAFADEQQPLNWVQTHDYSDELGTILSITIARKETI |
| | | DVNDRDAVTREVQKMFPGVEVLGTAAYDWTADPFSLGAWAA |
| | | YGVGQLSRLKDLQAAEGRILFAGAETSNGWHANIDGAVESGL |
| | | RAGREVKQLLS |
| NicA2Δ50 | 8 | GFDYDVVVVGGGFAGATAARECGLQGYRTLLLEARSRLGGRT |
| A107R | | FTSRFAGQEIEFGGRWVHWLQPHVWAEMQRYGLGVVEDPLT |
| (A107R | | NLDKTLEVIYNDGSVESISPDEFGKNIRIAFEKLCHDAWEVFPRP |
| substitution; | | HEPMFTERARELDKSSVLDRIKTLGLSRLQQAQINSYMALYAG |
| N-terminal | | ETTDKFGLPGVLKLFACGGWNYDAFMDTETHYRIQGGTIGLIN |
| deletion of | | AMLTDSGAEVRMSVPVTAVEQVNGGVKIKTDDDEIITAGVVV |
| residues 1 50) | | MTVPLNTYKHIGFTPALSKGKQRFIKEGQLSKGAKLYVHVKQN |
| | | LGRVFAFADEQQPLNWVQTHDYSDELGTILSITIARKETIDVND |
| | | RDAVTREVQKMFPGVEVLGTAAYDWTADPFSLGAWAAYGVG |
| | | QLSRLKDLQAAEGRILFAGAETSNGWHANIDGAVESGLRAGRE |
| | | VKQLLS |
| NicA2A107H | 9 | SDKTKTNEGFSRRSFIGSAAVVTAGVAGLGAIDAASATQKTNR |
| (A107H | | ASTVKGGFDYDVVVVGGGFAGATAARECGLQGYRTLLLEARS |
| substitution) | | RLGGRTFTSRFAGQEIEFGGHWVHWLQPHVWAEMQRYGLGV |
| | | VEDPLTNLDKTLIMYNDGSVESISPDEFGKNIRIAFEKLCHDAW |
| | | EVFPRPHEPMFTERARELDKSSVLDRIKTLGLSRLQQAQINSYM |
| | | ALYAGETTDKFGLPGVLKLFACGGWNYDAFMDTETHYRIQGG |
| | | TIGLINAMLTDSGAEVRMSVPVTAVEQVNGGVKIKTDDDEIITA |
| | | GVVVMTVPLNTYKHIGFTPALSKGKQRFIKEGQLSKGAKLYVH |
| | | VKQNLGRVFAFADEQQPLNWVQTHDYSDELGTILSITIARKETI |
| | | DVNDRDAVTREVQKMFPGVEVLGTAAYDWTADPFSLGAWAA |
| | | YGVGQLSRLKDLQAAEGRILFAGAETSNGWHANIDGAVESGL |
| | | RAGREVKQLLS |
| NicA2A107P | 10 | SDKTKTNEGFSRRSFIGSAAVVTAGVAGLGAIDAASATQKTNR |
| (A107P | | ASTVKGGFDYDVVVVGGGFAGATAARECGLQGYRTLLLEARS |
| substitution) | | RLGGRTFTSRFAGQEIEFGGPWVHWLQPHVWAEMQRYGLGV |
| | | VEDPLTNLDKTLIMYNDGSVESISPDEFGKNIRIAFEKLCHDAW |
| | | EVFPRPREPMFTERARELDKSSVLDRIKTLGLSRLQQAQINSYM |
| | | ALYAGETTDKFGLPGVLKLFACGGWNYDAFMDTETHYRIQGG |
| | | TIGLINAMLTDSGAEVRMSVPVTAVEQVNGGVKIKTDDDEIITA |
| | | GVVVMTVPLNTYKHIGFTPALSKGKQRFIKEGQLSKGAKLYVH |
| | | VKQNLGRVFAFADEQQPLNWVQTRDYSDELGTILSITIARKETI |
| | | DVNDRDAVTREVQKMFPGVEVLGTAAYDWTADPFSLGAWAA |
| | | YGVGQLSRLKDLQAAEGRILFAGAETSNGWHANIDGAVESGL |
| | | RAGREVKQLLS |

* The N-terminal methionine residue (M) of SEQ ID NO: 1 is cleaved off in the purified product; however all amino acid position designations disclosed herein take the methionine residue into account for the purpose of maintaining amino acid numbering conventions used in the art for the wild-type NicA2 sequence.
** Underlined sequences in wild-type NicA2 identify the six highest ranked immunogenic regions identified by the online MHC-II Binding Predictions tool on the Immune Epitope Database and Analysis Resource website (iedb.org) using the specific human MHC allele HLA DRB1*0401.
†Residues highlighted in grey were identified as MHCII epitopes.

As noted above the nicotine-degrading enzyme variants may exhibit increased nicotine-degrading activity and/or decreased immunogenicity relative to the wild-type NicA2. The variants may comprise one or more mutations to the amino acid sequence of wild-type NicA2, including one or more deletions, additions, or substitutions. A substitution mutation may be “conservative” or “non-conservative.” “Conservative” refers to a substitution within the same family of amino acids, while “non-conservative” refers to substitutions across families of amino acids. Families of amino acids and “conservative” and “non-conservative” substitutions relative thereto are known in the art. For example, the naturally occurring amino acids may be divided into the following four families and conservative substitutions will take place within those families, while non-conservative substitutions will take place across different families.
(1) Amino acids with basic side chains: lysine, arginine, histidine.
(2) Amino acids with acidic side chains: aspartic acid, glutamic acid
(3) Amino acids with uncharged polar side chains: asparagine, glutamine, serine, threonine, tyrosine.
(4) Amino acids with nonpolar side chains: glycine, alanine, valine, leucine, isoleucine, proline, phenylalanine, methionine, tryptophan, cysteine.

In some embodiments, the nicotine-degrading enzyme variants comprise one or more mutations in an active site of the wild-type NicA2 enzyme relevant to its nicotine-degrading activity, such as a mutation at one or more positions selected from any one of amino acid residues 90-93, 95, 102-109, 113, 116, 130, 132, 138, 155, 159, 210, 213-215, 217-220, 234, 245, 246, 248-251, 253, 254, 258, 334, 336, 339-342, 353, 355, 363-367, 378-382, 415-418, 423-429, 459-463, 465, or 466 of SEQ ID NO:1, such as one or more conservative substitutions, non-conservative substitutions, additions, or deletions in positions listed in Table 2. The Shell One residues identified in Table 2 make up the cavity surface, while the Shell Two residues contacting Shell One. For instance, in some embodiments, the disclosed nicotine-degrading enzyme variants can comprise at least one substitution at amino acid position 91, 104, 106, 107, 217, 250, 340, 355, 366, 381, 427, 462, or 463 of SEQ ID NO:1. In some embodiments, the variants may comprise one, two, or three or more substitutions.

TABLE 2
NicA2 Active Site Residues

| Shell One | Shell Two |
|---|---|
| ARG91 | GLY90 |
| PHE104 | THR92 |
| GLY105 | PHE93 |
| GLY106 | SER95 |
| ALA107 | ILE102 |
| TRP108 | GLU103 |
| TYR214 | VAL109 |
| TYR218 | GLN113 |
| GLU249 | VAL116 |
| THR250 | ASP130 |
| LYS340 | LEU132 |
| PHE355 | THR138 |
| TRP364 | PHE155 |
| GLN366 | ILE159 |
| THR381 | GLN210 |
| TRP417 | SER213 |
| ALA426 | MET215 |
| TRP427 | LEU217 |
| ALA461 | ALA219 |
| ASN462 | GLY220 |
| ILE463 | LEU234 |
| | PHE245 |
| | MET246 |
| | THR248 |
| | HIS251 |
| | ARG253 |
| | ILE254 |
| | THR258 |
| | GLN334 |
| | SER336 |
| | ALA339 |
| | LEU341 |
| | TYR342 |
| | PHE353 |
| | ASN363 |
| | VAL365 |
| | THR367 |
| | LEU378 |
| | SER379 |
| | ILE380 |
| | ILE382 |
| | TYR415 |
| | ASP416 |
| | THR418 |
| | SER423 |
| | LEU424 |
| | GLY425 |
| | ALA428 |
| | ALA429 |
| | TRP459 |
| | HIS460 |
| | GLY465 |
| | ALA466 |

Active site based on published crystal structure from Tararina et al., 2016.
Amino acid residue numbers correspond to SEQ ID NO: 1.

In some embodiments, at least one mutation that increases the nicotine-degrading activity or increases the catalytic activity of the enzyme is introduced into the variant, allowing the variant to more rapidly and/or more efficiently break-down nicotine. In some embodiments, such a mutation may improve various measures of enzymatic performance, including but not limited to, increasing k_{cat}, lowering K_{M}, increasing k_{cat}/K_{M} and/or increasing Vₘₐₓ. Thus, in some embodiments, a variant may comprise 1, 2, 3, 4, 5, 6, 7, 8, 9, or 10 or more mutations in an active site of the wild-type NicA2 or wild-type NOX enzymes and/or in the aromatic cage, and exhibit increased nicotine-degrading activity as measured by increased k_{cat}, lowered K_{M}, increased k_{cat}/K_{M}, and/or increased Vₘₐₓ, relative to the wild-type NicA2 enzyme.

In some embodiments, the nicotine-degrading enzyme variants comprise one or more mutations in the aromatic cage of the wild-type NicA2 enzyme formed by the tryptophan at position 427 and the asparagine at position 462 of SEQ ID NO:1, such as a mutation at one or more of these positions, such as one or more conservative substitutions, non-conservative substitutions, additions, or deletions. Thus, in some embodiments, a mutation that increases the nicotine-degrading activity is at one or more of positions 427 or 462 of SEQ ID NO:1, such as a conservative substitution, non-conservative substitution, addition, or deletion at one or more of positions 427 or 462 of SEQ ID NO:1.

In some embodiments, a mutation that increases the nicotine-degrading activity is a mutation that occurs at one or more of positions 91, 104, 106, 107, 217, 250, 340, 355, 366, 381, 427, 462, or 463 of SEQ ID NO:1, such as a conservative substitution, non-conservative substitution, addition, or deletion at one or more of positions 91, 104, 106, 107, 217, 250, 340, 355, 366, 381, 427, 462, or 463 of SEQ ID NO:1. Exemplary substitution mutations are shown in Table 1, and the disclosed variants may include at least one, at least two, at least 3, at least 4, or at least 5 of the substitutions disclosed in Tables 1, 2, or 3. Additionally or alternatively, the disclosed variants may also comprise a C-terminal or N-terminal deletion as discussed below.

Additionally or alternatively, in some embodiments, the nicotine-degrading enzyme variants comprise one or more mutations within an immunogenic T-cell epitope, such as one or more mutations within an immunogenic T-cell epitope within a region selected from positions 10-32, 68-94, 189-225, 248-285, 296-327, 336-391, or 435-459 of SEQ ID NO:1, such as one or more mutations within an immunogenic T-cell epitope selected from positions 16-24, 73-81, 258-266, 302-310, 373-381, or 447-455 of SEQ ID NO:1, such as one or more conservative substitutions, non-conservative substitutions, additions, or deletions in one or more of these regions. Thus, in some embodiments, a variant may comprise 1, 2, 3, 4, 5, 6, 7, 8, 9, or 10 or more mutations in 1, 2, 3, 4, 5, 6, or 7, immunogenic T-cells epitopes. In some embodiments, such variants exhibit reduced immunogenicity when administered to a mammalian subject.

In some embodiments, the nicotine-degrading enzymes variants comprise a mutation in an immunogenic T-cell epitope at one or more positions selected from 74, 77, 78, 80, 262-266, 303, 304, 306, 310, 374, 377, 378, 382, 383, 450-452, or 457 of SEQ ID NO:1, including all permutations and combinations thereof. For example, a variant may include any one or more of the mutations set forth below, including one or more of the exemplary mutations in Epitope B, one or more of the exemplary mutations in Epitope 1, one or more of the exemplary mutations in Epitope 2, one or more of the exemplary mutations in Epitope 3, and/or one or more of the exemplary mutations in Epitope 4. For instance, in some embodiments, the nicotine-degrading enzyme may have an amino acid substitution at position 262 and/or 263 of SEQ ID NO:1, such as an I262A substitution or I262T/N263R substitutions.

TABLE 3
Exemplary Mutations in the NicA2 Epitopes
(numbering based on SEQ ID NO: 1)

| Epitope B | Epitope 1 | Epitope 2 | Epitope 3 | Epitope 4 |
|---|---|---|---|---|
| L74N | I262A, | V303T, | L374Q, | I448Q, |
| Y77R | A264Q | V304N, | I377S | F450S |
| | | M306I | | |
| L74N, | I262K, | V304A, | L374A, | I448E, |
| Y77K | L266D | M306Q | I377A | F450N |
| L74Q, | I262T | V304A, | L374Q, | I448A, |
| Y77R | | M306N | I377A | F450N |
| L74Q, | I262S | V304A | L374N, | I448Q, |
| Y77N | | | I377A | F450Q |
| L74N, | I262D, | V304A, | L374N, | I448T, |
| Y77Q | L266K | M306H | I382Q | F450Q |
| L74N, | I262A | V304N, | I377A, | I448E, |
| Y77H | | M306H | I382T | F450L |
| L74N, | I262T, | V304Q, | I377A, | T455K |
| L80H | A264L | M306H | L378N | |
| L80F | I262T, | V304N, | I377T, | L449H, |
| | N263R | M306I | I382T | F450A |
| Y77R | M265H | V304T, | I377T | F450A |
| | | M306I | | |
| R78Q | I262A, | M306I, | L374N, | I448A, |
| | A264N | L310R | A383Q | F450Y |

Additionally or alternatively, in some embodiments, the nicotine-degrading enzyme variants comprise an N-terminal deletion of from 1 to 52 amino acid residues of SEQ ID NO:1. For example, in some embodiments a variant comprises an N-terminal deletion of amino acid residues 1-16, 1-25, 1-38, 1-50, 1-51, or 1-52 of SEQ ID NO:1. Thus, the disclosed variants may comprise an N-terminal deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 consecutive amino acids.

In some embodiments, the disclosed variants may additionally or alternatively comprise a deletion at the C-terminus of the peptide. For example, the disclosed variants may comprise a deletion of one or more amino acids at the C-terminus of the peptide. For example, in a NicA2 variant, the amino acid corresponding to 5482 of the wild-type sequence may be deleted.

In some embodiments, a nicotine-degrading enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the wild-type NicA2 enzyme (SEQ ID NO:1), or to an N-terminal deletion variant thereof having a deletion of up to 52 N-terminal amino acid residues of SEQ ID NO:1.

In some embodiments, a nicotine-degrading enzyme variant as described herein is or comprises SEQ ID NOs: 5-10. In some embodiments, a nicotine-degrading enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequences disclosed in Table 1.

In some embodiments, a variant as described herein exhibits increased nicotine-degrading activity relative to the wild-type NicA2 enzymes, such that its activity is at least about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2250%, about 2500%, about 2750%, about 3000%, about 3250%, about 3500%, about 3750%, about 4000%, about 4250%, about 4500%, about 4750%, or about 5000% or more than that of the wild-type NicA2 enzyme, as determined by an assay such as an AMPLEX® Red assay (Thermo Fisher Scientific).

In some embodiments, a variant as described herein exhibits increased nicotine-degrading activity relative to the wild-type NicA2 enzyme, such that its activity is at least about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2250%, about 2500%, about 2750%, about 3000%, about 3250%, about 3500%, about 3750%, about 4000%, about 4250%, about 4500%, about 4750%, or about 5000% or more than that of the wild-type NicA2 enzyme as determined by an assay where residual nicotine concentrations are measured using Gas Chromatography (GC; Hieda et al.: Immunization of rats reduces nicotine distribution to brain. Psychopharmacology, 143, 150-157, 1999) after incubation with a fixed concentration of enzyme in either buffer or rat serum at 37° C. and quenching activity at fixed time points by mixing with MeOH.

In some embodiments, a variant as described herein exhibits decreased immunogenicity in a mammalian subject relative to wild-type NicA2, such that it is at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% less immunogenic than the wild-type NicA2 enzyme. Unless otherwise specified, “decreased immunogenicity” as compared to the wild-type NicA2 enzyme as used herein refers decreased immunogenicity as shown by one or more of in silico approaches, in vitro assays, in vivo studies (e.g., using transgenic animals), ex vivo studies using human T-cells, or clinical studies with human subjects.

In some embodiments, the nicotine-degrading enzyme or variant thereof is a long-acting enzyme that has been modified in order to extend its half-life in vivo (after administration). Various techniques are known in the art for extending the circulating half-life of peptides. For example, in some embodiments the enzyme is conjugated to polyethylene glycol (PEG) or a similar polymer that prolongs half-life. Conjugating PEG to the disclosed nicotine-degrading enzyme or variant thereof can improve the pharmacokinetic properties of the variant. In some embodiments PEGylation has one or more effects selected from masking one or more immunogenic epitopes of the variant, decreasing variant-specific antibody titers, and attenuating T-cell proliferation and/or cytokine responses. Additionally or alternatively, in some embodiments, conjugating the variants to PEG does not decrease the enzymatic activity of the nicotine-degrading enzyme variants, or does not significantly decrease the enzymatic activity, or does not eliminate the enzymatic activity.

The PEG chain length and architecture (i.e., linear vs. branched) may be selected and varied to impact, impart, or promote different properties, as illustrated in the examples below. PEG can be conjugated to the variants by methods known for conjugating PEG to proteins, including those illustrated in the examples below. Any of the variants described herein can be PEGylated, including variants defined by or comprising any of, e.g., SEQ ID NOs: 1-10. For the purposes of conjugating PEG to the disclosed enzyme variants, the size or length of the PEG polymers can vary. For example, linear PEG conjugated to the disclosed enzyme variants may be in the range of 1-50 kDa, 5-40 kDa, or 10-20 kDa, such as 1, 2, 3, 4, 5, 6, 7, 8, 9, 10, 11, 12, 13, 14, 15, 16, 17, 18, 19, 20, 25, 30, 35, 40, 45, or 50 kDa. Additionally, the PEG polymers may be branched, with size in the range of 20-80 kDa, such as 20, 40, 60 or 80 kDa.

In some embodiments, the enzyme or variant thereof is fused to an albumin-binding peptide, an albumin-binding protein domain, human serum albumin, or an inert polypeptide. Exemplary inert polypeptides that have been used to increase the circulating half-life of peptides include, but are not limited to, XTEN® (also known as recombinant PEG or “rPEG”), a homo-amino acid polymer (HAP; HAPylation), a proline-alanine serine polymer (PAS; PASylation), or an elastin-like peptide (ELP; ELPylation). As used herein, “fused to” includes genetic fusion, directly or through a linker, resulting in a single polypeptide containing multiple domains, unless otherwise specified.

A nicotine-degrading enzyme used as disclosed herein can be formulated in a pharmaceutical composition suitable for administration to the target subject by the intended route of administration, as discussed in more detail below.

### VII. Nicotine-Binding Antibodies

In some embodiments, the anti-nicotine agent is a nicotine-binding antibody, a nicotine-binding fragment thereof, or a related construct capable of binding nicotine. For convenience, these agents are referred to collectively herein as “nicotine-binding antibodies.”

Anti-nicotine antibodies have been previously developed, primarily for the purpose of facilitating smoking cessation. See, e.g., WO 2002/058635; WO 2000/032239; WO 2003/082329; U.S. Patent Application Publication 2006/111271; U.S. Pat. Nos, 8,344,111; 8,232,072; 6,232,082; 7,547,712; 7,446,205; and Carrera et al., “Investigations using immunization to attenuate the psychoactive effects of nicotine,” Bioorg Med Chem 12(3):563-70 (2004). These patents, applications, and non-patent literature are incorporated by reference herein to the extent that they relate to anti-nicotine antibodies and related constructs, including nicotine-binding antibody fragments.

For the purposes of developing a nicotine-binding antibody, nicotine may be coupled to an immunogenic carrier, such as an immunogenic protein, to elicit an immune response and induce the production of nicotine-binding antibodies. General techniques for making antibodies can be employed. See, e.g., Kohler and Milstein, Eur. J. Immunol., 5: 511-519 (1976); Harlow and Lane (eds.), ANTIBODIES: A LABORATORY MANUAL, CSH Press (1988); C. A. Janeway et al. (eds.), IMMUNOBIOLOGY, 5th Ed., Garland Publishing, New York, N.Y. (2001).

100801 Anti-nicotine antibodies useful in the disclosed methods can be obtained by any means, including via in vitro sources (e.g., a hybridoma or a cell line producing an antibody recombinantly) and in vivo sources (e.g., rodents, rabbits, humans, etc.). Human, partially humanized, fully humanized, and chimeric antibodies can be made by methods known in the art, such as using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9): 1117-25 (2005), and Lonberg, Handb. Exp. Pharmacol., 181: 69-97 (2008)).

Nicotine-binding antibodies useful in the methods disclosed herein may be monoclonal and/or recombinant. Monoclonal antibodies (mAbs) may obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with a nicotine antigen. Recombinant antibodies may be obtained by methods known in the art, for example, using phage display technologies, yeast surface display technologies (Chao et al., Nat. Protoc., 1(2): 755-68 (2006)), mammalian cell surface display technologies (Beerli et al., PNAS, 105(38): 14336-41 (2008), and/or expressing or co-expressing antibody polypeptides. Other techniques for making antibodies are known in the art, and can be used to obtain antibodies used in the methods described herein.

Typically, an antibody consists of four polypeptides: two identical copies of a heavy (H) chain polypeptide and two copies of a light (L) chain polypeptide. Typically, each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody.

The terms “antibody fragment” and “nicotine-binding fragment,” as used herein, refer to one or more portions of a nicotine-binding antibody that exhibits the ability to bind nicotine. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H} CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples include nicotine-binding domain immunoglobulin fusion proteins comprising (i) a nicotine-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain CH2 constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization. See, e.g., U.S. patent application Ser. No. 2003/0118592; U.S. Patent Application U.S. 2003/0133939.

In some embodiments, a nicotine-binding antibody used in the methods disclosed herein is a human IgG1 antibody or a human IgG4 antibody. In some embodiments, the nicotine-binding antibody is mammalian, human, humanized, or chimeric. In some embodiments, nicotine-binding antibodies used as disclosed herein comprise one or more mutations that make the antibody more suitable in a therapeutic context.

Heavy and light chain sequences of exemplary IgG1 nicotine-binding antibodies are disclosed in Table 4 below. Heavy and light chain sequences of exemplary IgG4 nicotine-binding antibodies are disclosed in Table 5 below.

TABLE 4
Heavy and Light Chain Sequences of IgG1
Nicotine-Binding Antibodies

| Antibody | | SEQ ID |
|---|---|---|
| Chain | Amino Acid Sequence | NO: |
| 8D1 | QVRLQESGPGLVKPSGTLSLTCAVSGGSIYSSNWWTWVRQPPG | 11 |
| Heavy | KGLEWVGEIHIRGTTYYNPSLNSRVTISLDKSNNQVSLRLTSVT | |
| | AADSAVYYCVSQEVGGPDLWGQGTLVTVSSASTKGPSVFPLA | |
| | PSSKSTSGGTAALGCLVKDYFPEPVTVSWNSGALTSGVHTFPA | |
| | VLQSSGLYSLSSVVTVPSSSLGTQTYICNVNHKPSNTKVDKKVE | |
| | PKSCDKTHTCPPCPAPELLGGPSVFLFPPKPKDTLMISRTPEVTC | |
| | VVVDVSEEEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK | |
| 8D1 | NFMLTQPHSVSESPGKTVTISCTRSGGSIATYYVQWYQQRPGS | 12 |
| Light | APTNVIYKYDQRPSGVPDRFSGSIDSSSNSASLTISGLKTEDEAD | |
| | YYCQSYDNNIQVFGGGTKLTVLGQPKAAPSVTLFPPSSEELQAN | |
| | KATLVCLISDFYPGAVTVAWKADGSPVKAGVETTKPSKQSNNKYAA | |
| | SSYLSLTPEQWKSHRSYSCQVTHEGSTVEKTVAPTECS | |
| 12F5 | QLQLQESGPGLVKPSETLSLICTVSGGSIRKNNEWWAWIRQAP | 13 |
| Heavy | GKGLEWIGSLSYTGRTVYNPSLKSRVTISTDTSETQFSLKVNSV | |
| | TAADTAVYYCARLSPFVGAAWWFDPWGQGTLVTVSSASTKG | |
| | PSVFPLAPSSKSTSGGTAALGCLVKDYFPEPVTVSWNSGALTSG | |
| | VHTFPAVLQSSGLYSLSSVVTVPSSSLGTQTYICNVNHKPSNTK | |
| | VDKKVEPKSCDKTHTCPPCPAPELLGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPAPIEKTISKA | |
| | KGQPREPQVYTLPPSRDELTKNQVSLTCLVKGFYPSDIAVEWES | |
| | NGQPENNYKTTPPVLDSDGSFFLYSKLTVDKSRWQQGNVFSCS | |
| | VMHEALHNHYTQKSLSLSPGK | |
| 12F5 | EVVLTQSPGTLSLSPGERATLSCRASQSVSSRYLAWYQQKPGQ | 14 |
| Light | APRLLIYGASSRAIGTPDRFSGSGSGTDFTLTISRLEPEDFAVYY | |
| | CQQYAYSPPAITFGGGTKVEIKRTVAAPSVFIFPPSDEQLKSGTAS | |
| | VVCLLNNFYPREAKVQWKVDNALQSGNSQESVTEQDSKDSTYSLSS | |
| | TLTLSKADYEKHKVYACEVTHQGLSSPVTKSFNRGEC | |
| 7A8 | QLQLQESGPGLLKPSETLSLTCTVSGGSVTTSPDWWAWLRQSP | 15 |
| Heavy | GKGLEWIGSVSYTGRTVYNPSLKSRVTISLDTSKNHLSLRMTSA | |
| | TAADTAVFYCARLTPIDRFSADYYVLDIWGQGATVTVSSAST | |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| 7A8 | EIVMTQSPATLSVSPGERATLSCRASQSISSNLAWFQHKPGQAP | 16 |
| Light | RLLIFRSSTRATGTPPRFSGSGSGTEFTLTISSLQSEDFAVYFCQH | |
| | YSYWPPLITFGQGTRLEIKRTVAAPSVFIFPPSDEQLKSGTASVVC | |
| | LLNNFYPREAKVQWKVDNALQSGNSQESVTEQDSKDSTYSLSSTLTL | |
| | SKADYEKHKVYACEVTHQGLSSPVTKSFNRGEC | |
| 5D1 | QLQLRESGPGLVKPSETLSLTCSVSGGSISSSSYYWGWIRQPPG | 17 |
| Heavy | KGLEWIGSIYYTGRTYYNPSLESRVTISVDTSKNQFSLKLSSVT | |
| | AADTAVYYCAGLHYSWSALGGYYFYGMDVWGQGTTVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPE | |
| | VTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| 5D1 | EIVLTQSPGTLSLSPGERATLSCRASQSVSSRDLVWYQQKPGQA | 18 |
| Light | PRLLIYGASTRATGIPDRFSGSGSGTDFTLTISRLEPEDFAVYYC | |
| | QKYGSSPPRITFGPGTKVDIKRTVAAPSVFIFPPSDEQLKSGTASV | |
| | VCLLNATFYPREAKVQWKVDNALQSGNSQESVTEQDSKDSTYSLSST | |
| | LTLSKADYEKHKVYACEVTHQGLSSPVTKSFNRGEC | |
| 5G4 | QLQLQESGPGLVKPSETLSLTCSVSGGSISSSSYYWGWSRQSPG | 19 |
| Heavy | KGLEWIASIYYSGSTYYNPSLKSRVTIFIDTSKNQFSLKLSSVTA | |
| | ADTAIYYCARVGTSAMSRAFDMWGQGTMVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCDKTHTCPPCPAPELLGGPSVFLEPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| 5G4 | DIVMTQSPLSLPVTPGEPASISCRSSQSLLQSNGYNYLDWYLQK | 20 |
| Light | PGQSPQLLIYLGSNRASGVPDRFSGSGSGTDFTLKISKVEAEDV | |
| | GVYFCMQALQIPWTFGQGTKVEIKRTVAAPSVFIFPPSDEQLKS | |
| | GTASVVCLLNNFYPREAKVQWKVDNALQSGNSQESVTEQDSKDSTY | |
| | SLSSTLTLSKADYEKHKVYACEVTHQGLSSPVTKSFNRGEC | |
| 5H1 | QVQLQESGPGLVKPSETLSLTCTVSGGSISRRNDYWAWIRQSP | 21 |
| Heavy | GKDLEWIGTISFSGSTFYNPSLKSRVTISADTFNNHFSLRLDAVA | |
| | AADTAVYYCARLSPFVGAAWWFDPWGPGTLVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKV | |
| | DKKVEPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAKG | |
| | QPREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| 5H1 | EIVLTQSPGTLSLSPGERATLSCRASQSLSSNYLGWYQQKPGQA | 22 |
| Light | PRLLIYGASNRATGIPDRFSGSGSGTDFTLTISRLEPEDFGVYYC | |
| | QRYGRSPPAITFGGGTKVEIKRTVAAPSVFIFPPSDEQLKSGTASV | |
| | VCLLNNFYPREAKVQWKVDNALQSGNSQESVTEQDSKDSTYSLSST | |
| | LTLSKADYEKHKVYACEVTHQGLSSPVTKSFNRGEC | |
| 15A4 | QLQLQESGPGLVKPSETLSLTCTASGGSITNNIDYWVWIRQPPG | 23 |
| Heavy | RGLEWIGTIYYSGSTFYNPSLKSRVTISVDTSNNQFSLNLNSMS | |
| | AADTAVYYCARLRYYYDSNGYLPYWIDSWGQGTLVTVSSAS | |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPS | |
| | NTKVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSRDELTKNQVSLTCLVKGFYPSDIAVE | |
| | WESNGQPENNYKTTPPVLDSDGSFFLYSKLTVDKSRWQQGNVF | |
| | SCSVMHEALHNHYTQKSLSLSPGK | |
| 15A4 | EIVLTQSPGTLSLSPGERATLSCRASQSISSSYLGWYQQKPGQAP | 24 |
| Light | RLLIYGASSRATGIPDRFSGSGSGTDFTLTISSLEPEDFAVYFCQL | |
| | YRRSPPRLTFGGGTKVEIKRTVAAPSVFIFPPSDEQLKSGTASVVC | |
| | LLNNFYPREAKVQWKVDNALQSGNSQESVTEQDSKDSTYSLSSTLTL | |
| | SKADYEKHKVYACEVTHQGLSSPVTKSFNRGEC | |
| 2E11 | QLQLQESGPGLVKPSESLSLTCTVSGGSIISNDYYWAWIRQSPG | 25 |
| Heavy | KGLEWIGSINYRGSTFYSPSLNSRVTTSVDTSKNQFFLKLTSVT | |
| | AADTAMYFCTRLHGRYRGVGRLAFDYWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| 2E11 | DIQMTQSPSTLSASVGDIVTITCRASQSIGDWLAWYQQKPGKA | 26 |
| Light | PKLLIYKASNLESGVPSRFSGSGSGTEFTLTISSLQSDDFATYYC | |
| | QQYDSYSVTFGQGTKVEIKGTVAAPSVFIFPPSDEQLKSGTASVV | |
| | CLLNNFYPREAKVQWKVDNALQSGNSQESVTEQDSKDSTYSLSSTL | |
| | TLSKADYEKHKVYACEVTHQGLSSPVTKSFNRGEC | |
| 13F7 | QVQLQEAGPGLVKPSETLSLTCTVSGGSINTRNYYWGWVRQP | 27 |
| Heavy | PGKGLEWIASVYYTGSTFYDPSLRSRVTISIDTPRNQFSLRVSSV | |
| | DAGDMGVYYCVRLDGGYNNGYYYYGMDVWGQGTSVTVSS | |
| | ASTKGPSVFPLAPSSKSTSGGTAALGCLVKDYFPEPVTVSWNSG | |
| | ALTSGVHTFPAVLQSSGLYSLSSVVTVPSSSLGTQTYICNVNHK | |
| | PSNTKVDKKVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSRDELTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| 13F7 | GVQMTQSPSTLSASVGERVTVTCRASRPISNWLSWYQQKPGR | 28 |
| Light | APKLLIYGTSTLESGVPSRFSGSGSGTEFTLTITNLQPDDFATYY | |
| | CQEHNLYTITFGPGTKVEIKRTVAAPSVFIFPPSDEQLKSGTASVV | |
| | CLLNNFYPREAKVQWKVDNALQSGNSQESVTEQDSKDSTYSLSSTL | |
| | TLSKADYEKHKVYACEVTHQGLSSPVTKSFNRGEC | |
| 8H5 | QLQLQESGPGLVKPSETLSLSCAVSGASIRSNTYYWGWIRQPPG | 29 |
| Heavy | RGLEWIGSISHRGDAHYSPSLKSPVTISVDTSKNEFSLKATSVTA | |
| | ADTAVYYCVSLAYSFSWNTYYFYGMDVWGHGITVTVSSAST | |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| 8H5 | DIVLTQSPGTLSLSPGEGATLSCRASQSVNSGYLAWYQQKPGQ | 30 |
| Light | PPRLLVFAASSRATGIADRFRGSGSGTDFTLTITRLEPEDFAVYY | |
| | CQLYGHSPARITFGQGTRLETKRTVAAPSVFIFPPSDEQLKSGTA | |
| | SVVCLLNNFYPREAKVQWKVDNALQSGNSQESVTEQDSKDSTYSLS | |
| | STLTLSKADYEKHKVYACEVTHQGLSSPVTKSFNRGEC | |

Heavy and light chain complementarity determining regions (CDRs) are shown in bold, underlined text. CDR annotation was made according to IMGT numbering.
Constant regions are denoted in italicized, underlined text.

TABLE 5
Heavy and Light Chain Sequences of IgG4
Nicotine-Binding Antibodies

| Antibody | | SEQ |
|---|---|---|
| Chain | Amino Acid Sequence | ID NO: |
| 5G4-IgG4 | QLQLQESGPGLVKPSETLSLTCSVSGGSISSSSYYWGWSRQSP | 31 |
| Heavy | GKGLEWIASIYYSGSTYYNPSLKSRVTIFIDTSKNQFSLKLSSVT | |
| | AADTAIYYCARVGTSAMSRAFDMWGQGTMVTVSSASTKGPS | |
| | VFPLAPCSRSTSESTAALGCLVKDYFPEPVTVSWNSGALTSGV | |
| | HTFPAVLQSSGLYSLSSVVTVPSSSLGTKTYTCNVDHKPSNTK | |
| | VDKRVESKYGPPCPPCPAPEFLGGPSVFLFPPKPKDTLMISRTPE | |
| | VTCVVVDVSQEDPEVQFNWYVDGVEVHNAKTKPREEQFNST | |
| | YRVVSVLTVLHQDWLNGKEYKCKVSNKGLPSSIEKTISKAKG | |
| | QPREPQVYTLPPSQEEMTKNQVSLTCLVKGFYPSDIAVEWESN | |
| | GQPENNYKTTPPVLDSDGSFFLYSRLTVDKSRWQEGNVFSCSV | |
| | MHEALHNHYTQKSLSLSLGK | |
| 5G4-IgG4 | DIVMTQSPLSLPVTPGEPASISCRSSQSLLQSNGYNYLDWYLQ | 32 |
| Light | KPGQSPQLLIYLGSNRASGVPDRFSGSGSGTDFTLKISKVEAED | |
| | VGVYFCMQALQIPWTFGQGTKVEIKRTVAAPSVFIFPPSDEQL | |
| | KSGTASVVCLLNATFYPREAKVQWKVDNALQSGNSQESVTEQDSKD | |
| | STYSLSSTLTLSKADYEKHKVYACEVTHQGLSSPVTKSFNRGEC | |
| 7A8-IgG4 | QLQLQESGPGLLKPSETLSLTCTVSGGSVTTSPDWWAWLRQS | 33 |
| Heavy | PGKGLEWIGSVSYTGRTVYNPSLKSRVTISLDTSKNHLSLRMT | |
| | SATAADTAVFYCARLTPIDRFSADYYVLDIWGQGATVTVSSA | |
| | STKGPSVFPLAPCSRSTSESTAALGCLVKDYFPEPVTVSWNSGA | |
| | LTSGVHTFPAVLQSSGLYSLSSVVTVPSSSLGTKTYTCNVDHKP | |
| | SNTKVDKRVESKYGPPCPPCPAPEFLGGPSVFLFPPKPKDTLMI | |
| | SRTPEVTCVVVDVSQEDPEVQFNWYVDGVEVHNAKTKPREE | |
| | QFNSTYRVVSVLTVLHQDWLNGKEYKCKVSNKGLPSSIEKTIS | |
| | KAKGQPREPQVYTLPPSQEEMTKNQVSLTCLVKGFYPSDIAVE | |
| | WESNGQPENNYKTTPPVLDSDGSFFLYSRLTVDKSRWQEGNV | |
| | FSCSVMHEALHNHYTQKSLSLSLGK | |
| 7A8-IgG4 | EIVMTQSPATLSVSPGERATLSCRASQSISSNLAWFQHKPGQAP | 34 |
| Light | RLLIFRSSTRATGTPPRFSGSGSGTEFTLTISSLQSEDFAVYFCQ | |
| | HYSYWPPLITFGQGTRLEIKRTVAAPSVFIFPPSDEQLKSGTASV | |
| | VCLLNATFYPREAKVQWKVDNALQSGNSQESVTEQDSKDSTYSLSST | |
| | LTLSKADYEKHKVYACEVTHQGLSSPVTKSFNRGEC | |
| 12F5-IgG4 | QLQLQESGPGLVKPSETLSLICTVSGGSIRKNNEWWAWIRQAP | 35 |
| Heavy | GKGLEWIGSLSYTGRTVYNPSLKSRVTISTDTSETQFSLKVNS | |
| | VTAADTAVYYCARLSPFVGAAWWFDPWGQGTLVTVSSAST | |
| | KGPSVFPLAPCSRSTSESTAALGCLVKDYFPEPVTVSWNSGAL | |
| | TSGVHTFPAVLQSSGLYSLSSVVTVPSSSLGTKTYTCNVDHKPS | |
| | NTKVDKRVESKYGPPCPPCPAPEFLGGPSVFLFPPKPKDTLMIS | |
| | RTPEVTCVVVDVSQEDPEVQFNWYVDGVEVHNAKTKPREEQ | |
| | FNSTYRVVSVLTVLHQDWLNGKEYKCKVSNKGLPSSIEKTISK | |
| | AKGQPREPQVYTLPPSQEEMTKNQVSLTCLVKGFYPSDIAVEW | |
| | ESNGQPENNYKTTPPVLDSDGSFFLYSRLTVDKSRWQEGNVFS | |
| | CSVMHEALHNHYTQKSLSLSLGK | |
| 12F5-IgG4 | EVVLTQSPGTLSLSPGERATLSCRASQSVSSRYLAWYQQKPGQ | 36 |
| Light | APRLLIYGASSRAIGTPDRFSGSGSGTDFTLTISRLEPEDFAVYY | |
| | CQQYAYSPPAITFGGGTKVEIKRTVAAPSVFIFPPSDEQLKSGTA | |
| | SVVCLLNNFYPREAKVQWKVDNALQSGNSQESVTEQDSKDSTYSLS | |
| | STLTLSKADYEKHKVYACEVTHQGLSSPVTKSFNRGEC | |
| 8D1-IgG4 | QVRLQESGPGLVKPSGTLSLTCAVSGGSIYSSNWWTWVRQPP | 37 |
| Heavy | GKGLEWVGEIHIRGTTYYNPSLNSRVTISLDKSNNQVSLRLTS | |
| | VTAADSAVYYCVSQEVGGPDLWGQGTLVTVSSASTKGPSVF | |
| | PLAPCSRSTSESTAALGCLVKDYFPEPVTVSWNSGALTSGVHT | |
| | FPAVLQSSGLYSLSSVVTVPSSSLGTKTYTCNVDHKPSNTKVD | |
| | KRVESKYGPPCPPCPAPEFLGGPSVFLFPPKPKDTLMISRTPEVT | |
| | CVVVDVSQEDPEVQFNWYVDGVEVHNAKTKPREEQFNSTYR | |
| | VVSVLTVLHQDWLNGKEYKCKVSNKGLPSSIEKTISKAKGQPR | |
| | EPQVYTLPPSQEEMTKNQVSLTCLVKGFYPSDIAVEWESNGQP | |
| | ENNYKTTPPVLDSDGSFFLYSRLTVDKSRWQEGNVFSCSVMH | |
| | EALHNHYTQKSLSLSLGK | |
| 8D1-IgG4 | NFMLTQPHSVSESPGKTVTISCTRSGGSIATYYVQWYQQRPGS | 38 |
| Light | APTNVIYKYDQRPSGVPDRFSGSIDSSSNSASLTISGLKTEDEAD | |
| | YYCQSYDNNIQVFGGGTKLTVLGQPKAAPSVTLFPPSSEELQAN | |
| | KATLVCLISDFYPGAVTVAWKADGSPVKAGVETTKPSKQSNNKYAA | |
| | SSYLSLTPEQWKSHRSYSCQVTHEGSTVEKTVAPTECS | |
| 5D1-IgG4 | QLQLRESGPGLVKPSETLSLTCSVSGGSISSSSYYWGWIRQPPG | 39 |
| Heavy | KGLEWIGSIYYTGRTYYNPSLESRVTISVDTSKNQFSLKLSSVT | |
| | AADTAVYYCAGLHYSWSALGGYYFYGMDVWGQGTTVTVS | |
| | SASTKGPSVFPLAPCSRSTSESTAALGCLVKDYFPEPVTVSWNSGAL | |
| | TSGVHTFPAVLQSSGLYSLSSVVTVPSSSLGTKTYTCNVDHKPSNTK | |
| | VDKRVESKYGPPCPPCPAPEFLGGPSVFLFPPKPKDTLMISRTPEV | |
| | TCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYT | |
| | LPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKS | |
| | LSLSLGK | |
| 5D1-IgG4 | EIVLTQSPGTLSLSPGERATLSCRASQSVSSRDLVWYQQKPGQ | 40 |
| Light | APRLLIYGASTRATGIPDRFSGSGSGTDFTLTISRLEPEDFAVYY | |
| | CQKYGSSPPRITFGPGTKVDIKRTVAAPSVFIFPPSDEQLKSGTA | |
| | SVVCLLNNFYPREAKVQWKVDNALQSGNSQESVTEQDSKDSTYSLS | |
| | STLTLSKADYEKHKVYACEVTHQGLSSPVTKSFNRGEC | |
| 5H1-IgG4 | QVQLQESGPGLVKPSETLSLTCTVSGGSISRRNDYWAWIRQSP | 41 |
| Heavy | GKDLEWIGTISFSGSTFYNPSLKSRVTISADTFNNHFSLRLDAV | |
| | AAADTAVYYCARLSPFVGAAWWFDPWGPGTLVTVSSASTK | |
| | GPSVFPLAPCSRSTSESTAALGCLVKDYFPEPVTVSWNSGALTS | |
| | GVHTFPAVLQSSGLYSLSSVVTVPSSSLGTKTYTCNVDHKPSN | |
| | TKVDKRVESKYGPPCPPCPAPEFLGGPSVFLFPPKPKDTLMISR | |
| | TPEVTCVVVDVSQEDPEVQFNWYVDGVEVHNAKTKPREEQF | |
| | NSTYRVVSVLTVLHQDWLNGKEYKCKVSNKGLPSSIEKTISKA | |
| | KGQPREPQVYTLPPSQEEMTKNQVSLTCLVKGFYPSDIAVEWE | |
| | SNGQPENNYKTTPPVLDSDGSFFLYSRLTVDKSRWQEGNVFSC | |
| | SVMHEALHNHYTQKSLSLSLGK | |
| 5H1-IgG4 | EIVLTQSPGTLSLSPGERATLSCRASQSLSSNYLGWYQQKPGQ | 42 |
| Light | APRLLIYGASNRATGIPDRFSGSGSGTDFTLTISRLEPEDFGVYY | |
| | CQRYGRSPPAITFGGGTKVEIKRTVAAPSVFIFPPSDEQLKSGTA | |
| | SVVCLLNNFYPREAKVQWKVDNALQSGNSQESVTEQDSKDSTYSLS | |
| | STLTLSKADYEKHKVYACEVTHQGLSSPVTKSFNRGEC | |
| 15A4-IgG4 | QLQLQESGPGLVKPSETLSLTCTASGGSITNNIDYWVWIRQPP | 43 |
| Heavy | GRGLEWIGTIYYSGSTFYNPSLKSRVTISVDTSNNQFSLNLNSM | |
| | SAADTAVYYCARLRYYYDSNGYLPYWIDSWGQGTLVTVSSA | |
| | STKGPSVFPLAPCSRSTSESTAALGCLVKDYFPEPVTVSWNSGA | |
| | LTSGVHTFPAVLQSSGLYSLSSVVTVPSSSLGTKTYTCNVDHKP | |
| | SNTKVDKRVESKYGPPCPPCPAPEFLGGPSVFLFPPKPKDTLMI | |
| | SRTPEVTCVVVDVSQEDPEVQFNWYVDGVEVHNAKTKPREE | |
| | QFNSTYRVVSVLTVLHQDWLNGKEYKCKVSNKGLPSSIEKTIS | |
| | KAKGQPREPQVYTLPPSQEEMTKNQVSLTCLVKGFYPSDIAVE | |
| | WESNGQPENNYKTTPPVLDSDGSFFLYSRLTVDKSRWQEGNV | |
| | FSCSVMHEALHNHYTQKSLSLSLGK | |
| 15A4-IgG4 | EIVLTQSPGTLSLSPGERATLSCRASQSISSSYLGWYQQKPGQA | 44 |
| Light | PRLLIYGASSRATGIPDRFSGSGSGTDFTLTISSLEPEDFAVYFC | |
| | QLYRRSPPRLTFGGGTKVEIKRTVAAPSVFIFPPSDEQLKSGTAS | |
| | VVCLLNATFYPREAKVQWKVDNALQSGNSQESVTEQDSKDSTYSLSS | |
| | TLTLSKADYEKHKVYACEVTHQGLSSPVTKSFNRGEC | |
| 2E11-IgG4 | QLQLQESGPGLVKPSESLSLTCTVSGGSIISNDYYWAWIRQSPG | 45 |
| Heavy | KGLEWIGSINYRGSTFYSPSLNSRVTTSVDTSKNQFFLKLTSVT | |
| | AADTAMYFCTRLHGRYRGVGRLAFDYWGQGTLVTVSSAST | |
| | KGPSVFPLAPCSRSTSESTAALGCLVKDYFPEPVTVSWNSGAL | |
| | TSGVHTFPAVLQSSGLYSLSSVVTVPSSSLGTKTYTCNVDHKPS | |
| | NTKVDKRVESKYGPPCPPCPAPEFLGGPSVFLFPPKPKDTLMIS | |
| | RTPEVTCVVVDVSQEDPEVQFNWYVDGVEVHNAKTKPREEQ | |
| | FNSTYRVVSVLTVLHQDWLNGKEYKCKVSNKGLPSSIEKTISK | |
| | AKGQPREPQVYTLPPSQEEMTKNQVSLTCLVKGFYPSDIAVEW | |
| | ESNGQPENNYKTTPPVLDSDGSFFLYSRLTVDKSRWQEGNVFS | |
| | CSVMHEALHNHYTQKSLSLSLGK | |
| 2E11-IgG4 | DIQMTQSPSTLSASVGDIVTITCRASQSIGDWLAWYQQKPGKA | 46 |
| Light | PKLLIYKASNLESGVPSRFSGSGSGTEFTLTISSLQSDDFATYYC | |
| | QQYDSYSVTFGQGTKVEIKGTVAAPSVFIFPPSDEQLKSGTASVV | |
| | CLLNNFYPREAKVQWKVDNALQSGNSQESVTEQDSKDSTYSLSSTL | |
| | TLSKADYEKHKVYACEVTHQGLSSPVTKSFNRGEC | |
| 13F7-IgG4 | QVQLQEAGPGLVKPSETLSLTCTVSGGSINTRNYYWGWVRQP | 47 |
| Heavy | PGKGLEWIASVYYTGSTFYDPSLRSRVTISIDTPRNQFSLRVSS | |
| | VDAGDMGVYYCVRLDGGYNNGYYYYGMDVWGQGTSVTVS | |
| | SASTKGPSVFPLAPCSRSTSESTAALGCLVKDYFPEPVTVSWNS | |
| | GALTSGVHTFPAVLQSSGLYSLSSVVTVPSSSLGTKTYTCNVD | |
| | HKPSNTKVDKRVESKYGPPCPPCPAPEFLGGPSVFLFPPKPKDT | |
| | LMISRTPEVTCVVVDVSQEDPEVQFNWYVDGVEVHNAKTKPR | |
| | EEQFNSTYRVVSVLTVLHQDWLNGKEYKCKVSNKGLPSSIEK | |
| | TISKAKGQPREPQVYTLPPSQEEMTKNQVSLTCLVKGFYPSDIA | |
| | VEWESNGQPENNYKTTPPVLDSDGSFFLYSRLTVDKSRWQEG | |
| | NVFSCSVMHEALHNHYTQKSLSLSLGK | |
| 13F7-IgG4 | GVQMTQSPSTLSASVGERVTVTCRASRPISNWLSWYQQKPGR | 48 |
| Light | APKLLIYGTSTLESGVPSRFSGSGSGTEFTLTITNLQPDDFATYY | |
| | CQEHNLYTITFGPGTKVEIKRTVAAPSVFIFPPSDEQLKSGTASV | |
| | VCLLNATFYPREAKVQWKVDNALQSGNSQESVTEQDSKDSTYSLSST | |
| | LTLSKADYEKHKVYACEVTHQGLSSPVTKSFNRGEC | |
| 8H5-IgG4 | QLQLQESGPGLVKPSETLSLSCAVSGASIRSNTYYWGWIRQPP | 49 |
| Heavy | GRGLEWIGSISHRGDAHYSPSLKSPVTISVDTSKNEFSLKATSV | |
| | TAADTAVYYCVSLAYSFSWNTYYFYGMDVWGHGITVTVSSA | |
| | STKGPSVFPLAPCSRSTSESTAALGCLVKDYFPEPVTVSWNSGA | |
| | LTSGVHTFPAVLQSSGLYSLSSVVTVPSSSLGTKTYTCNVDHKP | |
| | SNTKVDKRVESKYGPPCPPCPAPEFLGGPSVFLFPPKPKDTLMI | |
| | SRTPEVTCVVVDVSQEDPEVQFNWYVDGVEVHNAKTKPREE | |
| | QFNSTYRVVSVLTVLHQDWLNGKEYKCKVSNKGLPSSIEKTIS | |
| | KAKGQPREPQVYTLPPSQEEMTKNQVSLTCLVKGFYPSDIAVE | |
| | WESNGQPENNYKTTPPVLDSDGSFFLYSRLTVDKSRWQEGNV | |
| | FSCSVMHEALHNHYTQKSLSLSLGK | |
| 8H5-IgG4 | DIVLTQSPGTLSLSPGEGATLSCRASQSVNSGYLAWYQQKPGQ | 50 |
| Light | PPRLLVFAASSRATGIADRFRGSGSGTDFTLTITRLEPEDFAVY | |
| | YCQLYGHSPARITFGQGTRLETKRTVAAPSVFIFPPSDEQLKSG | |
| | TASVVCLLNATFYPREAKVQWKVDNALQSGNSQESVTEQDSKDSTY | |
| | SLSSTLTLSKADYEKHKVYACEVTHQGLSSPVTKSFNRGEC | |

Heavy and light chain complementarity determining regions (CDRs) are shown in bold, underlined text. CDR annotation was made according to IMGT numbering.
Constant regions are denoted in italicized, underlined text.

Also useful in the methods disclosed herein are nicotine-binding antibodies and nicotine-binding fragments thereof comprising the same CDR sequences and/or the same framework region sequences and/or the same variable region sequences as one or more of the antibodies disclosed in Tables 4 and 5. In this regard, although the nicotine-binding antibodies disclosed in Tables 4 and 5 are IgG1 and IgG4 antibodies, respectively, other nicotine-binding antibodies within the scope of this disclosure may be IgG2, IgG3, IgA1, IgA2, IgE, IgH, or IgM, for example.

Human immunoglobulin IgG4 antibodies are good candidates for antibody-based therapy when, as here, reduced effector functions are desirable. However, IgG4 antibodies are dynamic molecules able to undergo a process known as Fab arm exchange (FAE). See, e.g., Labrijn et al., Therapeutic IgG4 antibodies engage in Fab-arm exchange with endogenous human IgG4 in vivo, NATURE BIOTECH 27(8): 767-71 (2009). This results in functionally monovalent, bispecific antibodies (bsAbs) with unknown specificity and hence, potentially, reduced therapeutic efficacy. FAE can be prevented by introducing a S228P mutation into the hinge region of the antibody. Thus, in some embodiments, a nicotine-binding antibody used as disclosed herein comprises a S228P substitution. The antibodies disclosed in Table 5 comprise such a S228P substitution. In other embodiments, a nicotine-binding antibody used as disclosed herein does not comprise a S228P substitution.

In some embodiments, a nicotine-binding antibody used as disclosed herein comprises one or more additional or alternative substitutions, insertions, or deletions beyond the aforementioned S228P substitution. For example, in some embodiments, a nicotine-binding antibody of the present disclosure comprises heavy and light chains with at least about 85%, about 86%, about 87%, about 88%, about 89%, about 90%, about 91%, about 92%, about 93%, about 94%, about 95%, about 96%, about 97%, about 98%, about 99%, or about 100% identity to one or more of the heavy and light chain sequences disclosed in Tables 4 and 5, respectively. In some embodiments, a nicotine-binding antibody of the present disclosure comprises heavy and light chains with at least 85%, 86%, 87%, 88%, 89%, 90%, 91%, 92%, 93%, 94%, 95%, 96%, 97%, 98%, 99%, or 100% identity to one or more of the heavy and light chain sequences disclosed in Tables 4 and 5, respectively.

Nicotine has two enantiomers: S-(−)-nicotine and R-(+)-nicotine, with the S-enantiomer known to be the most physiologically active. In some embodiments, the disclosed nicotine-binding antibodies exhibit selectivity for one enantiomer over the other. For instance, in some embodiments, a nicotine-binding antibody selectively binds to S-(−)-nicotine with a higher affinity than it binds to R-(+)-nicotine, while in some embodiments a nicotine-binding antibody may bind S-(−)-nicotine and substantially not bind to R-(+)-nicotine. For example, 8D1-IgG4 and 12F5-IgG4 preferentially bind to S-(−)-nicotine. In this regard, 8D1-IgG4 has a K_{D} for R-(+)-nicotine of 92 nM and 12F5-IgG4 has a K_{D} for R-(+)-nicotine of 1.2 μM. These disclosed antibodies exhibit greater binding affinity and selectivity for S-(−)-nicotine than has previously been reported for previously described nicotine-binding antibodies, such as the Nic12 mAb, which is disclosed in U.S. Pat. No. 8,344,111 and Tars et al., J. Mol. Bio., 415: 118-127 (2012). Alternatively, in some embodiments, a nicotine-binding antibody used as disclosed herein may selectively bind to R-(+)-nicotine with a higher affinity than it binds to S-(−)-nicotine, while in some embodiments a nicotine-binding antibody used as disclosed herein may bind to R-(+)-nicotine and substantially not bind to S-(−)-nicotine. In some embodiments, a nicotine-binding antibody used as disclosed herein may bind to both enantiomers of nicotine with comparable affinity.

In some embodiments, the nicotine-binding antibodies used as disclosed herein have a strong binding affinity for nicotine (one or both enantiomers) and a comparatively weak binding affinity for other molecules that may be present in a subject being treated, including molecules that are chemically- and/or structurally-related to nicotine, metabolites or byproducts of nicotine (e.g., cotinine), molecules that are ligands of or that bind to nicotinic receptors, drugs (e.g., small molecule drugs) used to aid smoking cessation (e.g., bupropion, varenicline, and cytisine) and/or treat nicotine addiction and/or nicotine toxicity, and/or other endogenous or exogenous molecules that may be present in a subject's blood, including neurotransmitters and other molecules that may be administered to diagnose or treat a condition in the subject or to maintain or support normal physiology. In other words, in some embodiments, the nicotine-binding antibodies used as disclosed herein do not cross-react with molecules that are not nicotine, i.e., “off-target compounds”.

In some embodiments, the nicotine-binding antibody or fragment used as disclosed herein is a long-acting variant that has been modified in order to extend its half-life in vivo (after administration). Various techniques are known in the art for extending the circulating half-life of peptides, such as antibodies. For example, in some embodiments the antibody carries mutations in the Fc region with enhanced FcRn-mediated recycling such as “YTE” (M252Y/S254T/T256E), see e.g., Dall'Acqua et al., J Biol Chem., 281:23514-24 (2006), or “Xtend” Fc domain mutations from Xencor (US 2014/0056879 A1). In other embodiments, the antibody or fragment thereof is conjugated to polyethylene glycol (PEG; i.e., the antibody is PEGylated) or a similar polymer that prolongs half-life. In some embodiments, the antibody is fused to an albumin-binding peptide, an albumin-binding protein domain, human serum albumin, or an inert polypeptide. Exemplary inert polypeptides that have been used to increase the circulating half-life of peptides include, but are not limited to, XTEN® (also known as recombinant PEG or “rPEG”), a homo-amino acid polymer (HAP; HAPylation), a proline-alanine serine polymer (PAS; PASylation), or an elastin-like peptide (ELP; ELPylation). As used herein, “fused to” includes genetic fusion, directly or through a linker, resulting in a single polypeptide containing multiple domains, unless otherwise specified.

A nicotine-binding antibody or a nicotine-binding fragment thereof used as disclosed herein can be formulated in a pharmaceutical composition suitable for administration to the target subject by the intended route of administration, as discussed in more detail below.

### VIII. Pharmaceutical Compositions

Pharmaceutical compositions suitable for use in the methods described herein can be formulated with an anti-nicotine agent as disclosed above and a pharmaceutically acceptable carrier or diluent.

The composition may be formulated for intravenous, subcutaneous, intraperitoneal, intramuscular, oral, nasal, pulmonary, ocular, vaginal, or rectal administration. In some embodiments, nicotine-binding antibodies are formulated for intravenous, subcutaneous, intraperitoneal, or intramuscular administration, such as in a solution, suspension, emulsion, liposome formulation, etc. In some embodiments, nicotine-degrading enzymes are formulated for intravenous, subcutaneous, intraperitoneal, or intramuscular administration, such as in a solution, suspension, emulsion, liposome formulation, etc. In some embodiments, the anti-nicotine agent may be formulated for administration by injection or infusion.

The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations (such as solid oral dosage forms are known); solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

### IX. Methods of Treating Cardiovascular Disease or Improving Circulation

As noted above, the methods of treating cardiovascular disease or improving circulation described herein comprise administering to a mammalian subject in need thereof an anti-nicotine agent, such as a nicotine-binding antibody or nicotine-binding fragment thereof or a nicotine-degrading enzyme or variant thereof as disclosed above. In some embodiments, the disclosed methods comprise administering a pharmaceutical composition comprising the anti-nicotine agent.

Without being bound by theory, it is believed that the presently disclosed methods function by blocking, neutralizing, and/or eliminating nicotine's physiological/pharmacological action. In other words, the disclosed methods reflect a “ligand-targeting” approach (i.e., binding/sequestering or degrading/removing nicotine) instead of antagonizing the nicotinic receptors. This results in blocking nicotine's actions on all nicotinic receptors, as opposed to small molecule antagonists, which possess specificity for a given nicotinic receptor or subtype. Thus, the presently disclosed methods function more efficiently and more broadly than previous approaches based on small molecule nicotine receptor antagonists.

In typical embodiments, the subject is a human. In some embodiments, the subject is suffering from a cardiovascular disease specifically associated with smoking or the use of nicotine or exposure to nicotine, such as Buerger's Disease, critical limb ischemia, peripheral artery disease, thrombus formation, and atherosclerotic disease. In some embodiments, the subject is at risk of developing a cardiovascular disease specifically associated with smoking, such as Buerger's Disease, critical limb ischemia, and peripheral artery disease.

In some embodiments, the subject is a tobacco user/nicotine consumer of some kind, e.g., a tobacco smoker or e-cigarette user (referred to collectively as a “smoker”). While smoking is the most common form of tobacco consumption, consumption of smokeless tobacco has been associated with the development of cardiovascular diseases like Buerger's Disease, critical limb ischemia, and peripheral artery disease. As noted above, the methods disclosed herein are effective for treating cardiovascular diseases, such as Buerger's Disease, critical limb ischemia, and peripheral artery disease, even when the subject has not ceased consumption of tobacco/nicotine products. In some embodiments, the subject is a current smoker/user of nicotine products, who may or may not be actively trying to quit or reduce consumption. In some embodiments, the subject is a former smoker/user of nicotine products.

In some embodiments, the disclosed methods of treatment are the only pharmaceutical treatment the subject is receiving for cardiovascular disease. In other embodiments, the subject may be treated with another pharmacological agent to address the symptoms and/or effects of the cardiovascular disease. For example, in some embodiments, the subject may be previously, concurrently, or subsequently treated with anti-platelet drugs, thrombolytic agents, or cilostazol. In some embodiments, the subject may have undergone or undergo vascular or endovacscular surgery (e.g., angioplasty, atherectomy, vascular bypass, or thrombectomy) and/or limb amputation.

In some embodiments, the subject is administered a therapeutically effective amount of the anti-nicotine agent, such as an amount effective to reduce plasma levels of nicotine, and/or reduce, ameliorate, or eliminate one or more symptoms or effects of a cardiovascular disease, such as Buerger's Disease, critical limb ischemia, peripheral artery disease, or atherosclerotic disease. Examples of symptoms or effects of a cardiovascular disease that may be reduced, ameliorated, or eliminated by the methods described herein include, but are not limited to, high blood pressure, high heart rate, and vasoconstriction. Other specific symptoms or effects of a cardiovascular disease that may be reduced, ameliorated, or eliminated by the methods described herein include, but are not limited to, acute or chronic inflammation and/or thrombosis of arteries and/or veins, including arteries and/or veins of the hands and/or feet.

In some embodiments, the subject is administered a therapeutically effective amount of the anti-nicotine agent, such as an amount effective to reduce plasma levels of nicotine, and/or reduce, ameliorate, or eliminate one or more symptoms or effects of a non-cardiovascular disease, such as cataracts, blindness (e.g., macular degeneration), excessive tearing, stinging of the eyes, stroke, nicotine addiction, cancer (e.g., cancer of the nasal cavity, paranasal sinus, lungs, lips, mouth, throat, larynx, pharynx, tracheal, esophageal, gastric, colon, pancreatic, breast, liver, prostate, bladder, kidney, ureter, cervical, ovarian, bone marrow, acute myeloid leukemia, etc.), chronic rhinosinusitis, impaired sense of smell, periodontal disease, dental decay, impaired sense of taste, hearing loss, ear infection, acute or chronic bronchitis, chronic obstructive pulmonary disease (COPD), emphysema, worsening of respiratory infections (tuberculosis, pneumonia, influenza), worsening of asthma, chronic cough, shortness of breath, excess sputum production, abdominal aortic aneurysm, peptic ulcer (esophagus, stomach, upper GI tract), reduced fertility, impotence, premature ovarian failure, early menopause, painful menstruation, Reynaud's disease, poor circulation, wrinkling, premature aging, loss of skin tone, osteoporosis, bone fracture (e.g., hip, knee and spinal fracture), rheumatoid arthritis, back problems; impaired wound healing, poor post-surgical recovery, leg pain, cold feet, gangrene, deep vein thrombosis, impaired resistance to infection, increased risk of allergic disorders, increased risk of diabetes, diabetic nephropathy, diabetic skin ulcers diabetic circulatory disorders, neuropathy, diabetes retinal disorders, diabetes related ulceration, vasculitis, amputation, and sudden death.

The disclosed methods may involve administering a therapeutically effective amount of an anti-nicotine agent (e.g., a nicotine-binding antibody, a nicotine-degrading enzyme, or a pharmaceutical composition comprising the same) to the subject. In some embodiments, the methods comprise administering a nucleic acid encoding the anti-nicotine agent (e.g., encoding one of the disclosed nicotine-binding antibodies or nicotine-degrading enzymes) in a construct that expresses the antibody or enzyme in vivo. For example, in such embodiments, the nucleic acid can be provided in a suitable vector, such as an adeno-associated virus (AAV) gene transfer vector. Other exemplary vectors that are suitable for use in such methods are known in the art. See, e.g., Lukashev and Zamyatnin, Biochem., 81(7): 700-8 (2016)). Exemplary vectors may include one or more enhancers (e.g., a cytomegalovirus (CMV) enhancer), promoters (e.g., chicken β-actin promoter), and/or other elements enhancing the properties of the expression cassette. Methods of making suitable vectors and general methods of using expression vectors in vivo are known in the art. See, e.g., Hicks et al., Sci. Transl. Med., 4(140): 140ra87 (2012). Accordingly, in some aspects, provided herein are methods of expressing a nicotine-binding antibody and or nicotine-degrading enzyme in vivo in a subject in need thereof, comprising administering to the subject a therapeutically effective amount of an expression vector capable of expressing an anti-nicotine antibody or nicotine-degrading enzyme in vivo.

The specific amount administered may depend on one or more of the amount and pattern of nicotine consumption by the subject, age and/or weight of the subject, the particular condition being treated (e.g., Buerger's disease versus critical limb ischemia), the severity of the condition being treated, and the localization of the underlying pathophysiology (e.g., peripheral artery disease in the legs versus peripheral artery disease of the kidneys).

In some embodiments, a nicotine-binding antibody may be administered at a dose of from about 10 to about 1000 mg/kg, about 15 mg/kg to about 850 mg/kg, about 20 mg/kg to about 750 mg/kg, about 30 mg/kg to about 650 mg/kg, about 40 mg/kg to about 550 mg/kg, about 50 mg/kg to about 450 mg/kg, about 60 mg/kg to about 350 mg/kg, about 70 mg/kg to about 250 mg/kg, or about 80 mg/kg to about 150 mg/kg. In some embodiments, the nicotine-binding antibody is administered at a dose of from about 20 mg/kg to about 80 mg/kg. In some embodiments, the nicotine-binding antibody is administered at a dose of about 10 mg/kg, about 15 mg/kg, about 20 mg/kg, about 25 mg/kg, about 30 mg/kg, about 35 mg/ kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600, about 650 mg/kg, about 700 mg/kg, about 750 mg/kg, about 800 mg/kg, about 850 mg/kg, about 900 mg/kg, about 950 mg/kg, or about 1000 mg/kg. In some embodiments, the nicotine-binding antibody is administered at a dose of 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600, 650 mg/kg, 700 mg/kg, 750 mg/kg, 800 mg/kg, 850 mg/kg, 900 mg/kg, 950 mg/kg, or 1000 mg/kg. In some embodiments, the nicotine-binding antibody is administered at a dose of about 3000 mg, about 3500 mg, about 4000 mg, about 4500 mg, about 5000 mg, about 5500 mg, about 6000, about 6500 mg, about 7000 mg, about 7500 mg, about 8000 mg, about 8500 mg, about 9000 mg, about 9500 mg, about 10000 mg, about 10500 mg, about 11000 mg, about 11500 mg, or about 12000 mg. In some embodiments, the nicotine-binding antibody is administered at a dose of 3000 mg, 3500 mg, 4000 mg, 4500 mg, 5000 mg, 5500 mg, 6000, 6500 mg, 7000 mg, 7500 mg, 8000 mg, 8500 mg, 9000 mg, 9500 mg, 10000 mg, 10500 mg, 11000 mg, 11500 mg, or 12000 mg. In some embodiments, the nicotine-binding antibody is administered at a dose of up to about 10 g. When other antibody-related constructs are used, such as antibody fragments, they can be used at comparable doses adjusted for their different molecular weights and/or binding affinities. For example, the dose of a fragment can be chosen to achieve comparable Cmax and/or AUC parameters as the corresponding full-length antibody, or to achieve binding of a comparable amount of nicotine. In some embodiments, more than one antibody may be administered, and when more than one antibody is administered, the total amount of antibody administered may be in accordance with the foregoing guidance.

In some embodiments, a nicotine-degrading enzyme may be administered at a dose of from about 0.01 to about 30 mg/kg, about 0.1 mg/kg to about 25 mg/kg, about 1 mg/kg to about 20 mg/kg, about 2 mg/kg to about 15 mg/kg, or about 5 mg/kg to about 10 mg/kg. In some embodiments, a variant is administered at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8/5 mg/kg, about 9 mg/kg, about 9.5 mg/kg, about 10 mg/kg, about 10.5 mg/kg, about 12 mg/kg, about 12.5 mg/kg, about 13 mg/kg, about 13.5 mg/kg, about 14 mg/kg, about 14.5 mg/kg, about 15 mg/kg, about 15.5 mg/kg, about 16 mg/kg, about 16.5 mg/kg, about 17 mg/kg, about 17.5 mg/kg, about 18 mg/kg, about 18.5 mg/kg, about 19 mg/kg, about 19.5 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In some embodiments, a nicotine-degrading enzyme or variant is administered at a dose of about 0.5 mg, about 1 mg, about 2.5 mg, about 5 mg, about 10 mg, about 15 mg, about 20 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100, about 2150 mg, about 2200 mg, about 2250 mg, about 2300 mg, about 2350 mg, about 2400 mg, about 2450 mg, or about 2500 mg. In some embodiments, more than one enzyme or variant may be administered, and when more than one enzyme is administered, the total amount administered may be in accordance with the foregoing guidance.

As noted above, the anti-nicotine agent may be administered by any suitable route of administration, such as via an injection, such as intravenously, subcutaneously, intramuscularly, or intraperitoneally.

In some embodiments, the method comprises administering a single dose of a pharmaceutical composition comprising an anti-nicotine agent or a single dose of a pharmaceutical composition comprising an anti-nicotine agent and another pharmaceutical compound, such as another compound for treating the cardiovascular disease. In other embodiments, the method comprises administering repeated doses of the pharmaceutical composition(s). In some embodiments, treatment is continued until one or more symptoms or effects of the cardiovascular disease are reduced, ameliorated, or eliminated. For instance, a subject with Buerger's Disease may be evaluated for the presence and/or severity of signs and symptoms associated with the disease, including, but not limited to, acute and chronic inflammation and thrombosis of arteries and veins of the hands and feet, and treated with one or more pharmaceutical composition(s) as described herein until one or more of the signs/symptoms is reduced, ameliorated, or eliminated after treatment. Additionally or alternatively, other symptoms or effects, such as blood pressure, heart rate, and vasoconstriction, which may be measured by skin temperature, may be used to determine the effectiveness and/or adequacy of a given dose or dosing regimen. Additionally, or alternatively, nicotine plasma levels may be detected or monitored to assess efficacy and/or adequacy. Additionally, or alternatively, in embodiments in which the subject is administered a nicotine-binding antibody or fragment thereof, the proportion of antibody-bound nicotine compared to unbound (free) nicotine in circulation may be compared.

In some embodiments, the methods comprise administering an anti-nicotine agent three or more times a day, twice a day, or once a day. In some embodiments, the methods comprise administering an anti-nicotine agent once a day, once every other day, three times a week, twice a week, once a week, once every other week, once every three weeks, once a month, or less frequently. In such embodiments, the anti-nicotine agent may be a long-acting agent, such as an agent constructed to have a long circulating half-life (e.g., via PEGylation). Additionally or alternatively, the composition may be an extended release composition.

In some embodiments, treatment may continue for 1, 2, 3, 4, 5, 6, 7, 8, 9, 10, 11, 12, 13, 14, 15, 16, 17, 18, 19, 20, or 21 or more days; 1, 2, 3, 4, 5, 6, 7, 8, 9, 10, 11, 12, 13, 14, 15, 16, 17, or 18 or weeks months; or 1, 2, 3, 4, 5, 6, 7, 8, 9, 10, 11, or 12 or more months; or 1, 2, or 3 or more years or until the signs, symptoms, and/or effects of the cardiovascular disease are reduced, eliminated, or ameliorated.

The following examples illustrate the invention. It should be understood, however, that the invention is not to be limited to the specific conditions or details described in these examples. All printed publications referenced herein are specifically incorporated by reference.

### X. Examples

### Example 1—Anti-Nicotine Activity of Anti-Nicotine Agents

This example illustrates the anti-nicotine activity of anti-nicotine agents as descried herein.

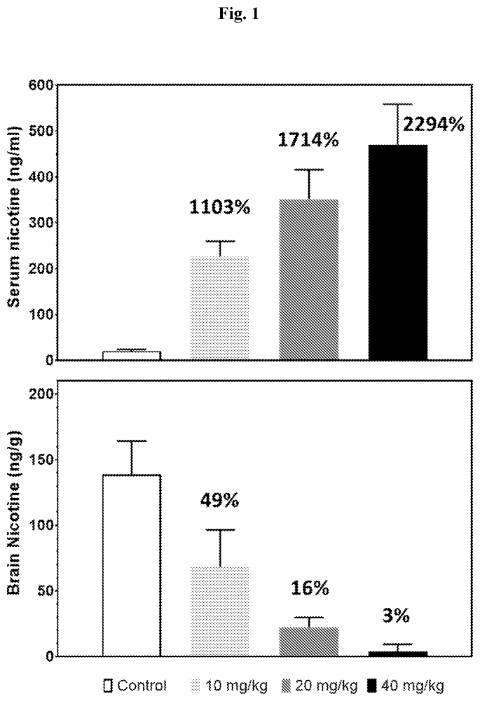

A single dose of 0.03 mg/kg nicotine (equal to roughly 2 cigarettes) was administered to rats (n=8) following pretreatment with either control (vehicle) or 10, 20, or 40 mg/kg of an anti-nicotine antibody, 8D1-IgG4. The nicotine was administered in <10 seconds as compared to the roughly 10 minutes required to smoke a cigarette. Animals were sacrificed after 3 minutes, and as shown in FIG. 1, there was greater than a 95% reduction in nicotine levels in the brain at the highest dose.

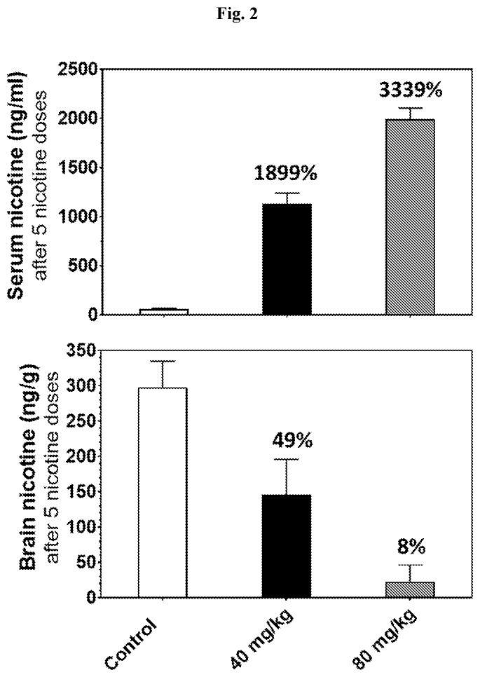

In a follow-up study using the 40 and 80 mg/kg antibody doses and the same nicotine dose, a >90% reduction of nicotine distribution to the brain was maintained even after repeated doses of nicotine (5 repeated doses, one every 10 min for 50 min) simulating very heavy smoking, as shown in FIG. 2. Indeed, at an 80 mg/kg dose of the antibody, brain nicotine levels were reduced by 92% 3 minutes after the fifth dose of nicotine.

A single dose of 0.03 mg/kg nicotine (equal to roughly 2 cigarettes) was administered to rats (n=8) following pretreatment with either control (vehicle) or 10, 20, or 40 mg/kg of an anti-nicotine antibody, 8D1-IgG4. The nicotine was administered in <10 seconds as compared to the roughly 10 minutes required to smoke a cigarette. Animals were sacrificed after 3 minutes, and as shown in FIG. 1, there was greater than a 95% reduction in nicotine levels in the brain at the highest dose.

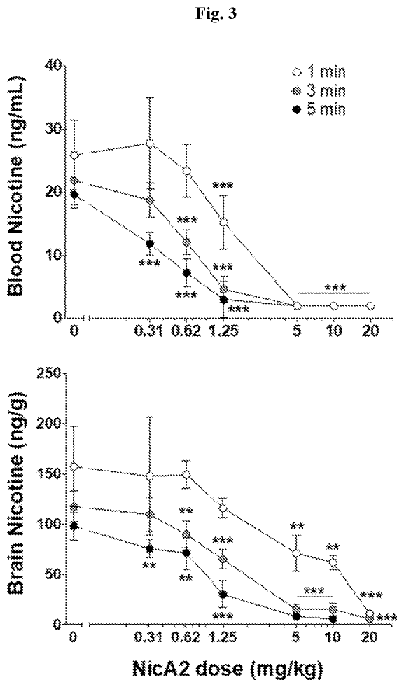

A single dose of 0.03 mg/kg nicotine (equal to roughly 2 cigarettes) was administered to rats following pretreatment with either control (vehicle) or a range of doses of the nicotine-degrading enzyme NicA2 on nicotine distribution to blood and brain in rats, over periods of 1, 3 or 5 min, were analyzed and are shown in FIG. 3. The nicotine was administered in <10 seconds as compared to the roughly 10 minutes required to smoke a cigarette. The effects of NicA2 were dose and time dependent (p<0.0001 by 2-way ANOVA). NicA2 effects on blood or brain nicotine concentrations were substantial even at 1 min but were greater, particularly in brain, at 5 min. Blood nicotine levels were significantly lower than in controls at all sampling times in groups receiving NicA2 at doses ≥1.25 mg/kg and were reduced to <2 ng/ml in all 64 rats receiving NicA2 doses of ≥5 mg/kg. For rats receiving ≥5 mg/kg NicA2 the blood nicotine level was reduced by >90% at all sampling intervals compared to controls. NicA2 efficacy in reducing brain nicotine levels was greater at 5 min than at earlier intervals. Brain nicotine levels were significantly lower than controls at 5 min in all groups receiving ≥0.31 mg/kg NicA2, at 3 min in groups receiving ≥0.62 mg/kg NicA2, and at 1 min in rats receiving ≥5 mg/kg NicA2. Although ≥5 mg/kg NicA2 reduced brain nicotine levels by 95% at 3 and 5 min, a higher dose of 20 mg/kg dose was needed to reduce brain nicotine levels to the same extent at one minute.

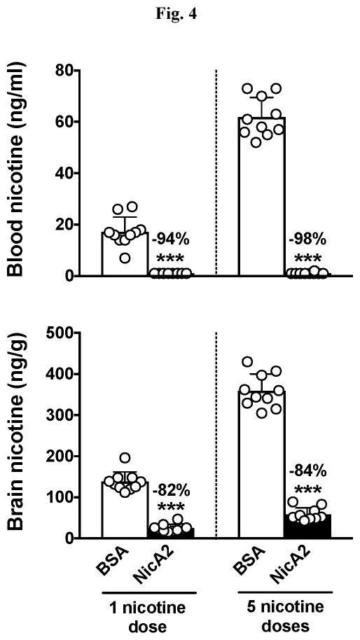

In a follow-up study using 10 mg/kg NicA2 administered i.v. and the same nicotine dose, nicotine concentrations in blood and brain were significantly and substantially lower than controls in NicA2-treated rats receiving either a single nicotine dose or a series of 5 nicotine doses (FIG. 4). Blood nicotine concentrations were below the limit of assay detection for most rats receiving NicA2. Brain nicotine concentrations were reduced in rats receiving NicA2 by 82% after the single nicotine dose and by 84% after the series of 5 nicotine doses, compared to their controls. These studies show that the disclosed anti-nicotine agents are capable of reducing nicotine distribution to the brain.

### Example 2—Efficacy of Anti-Nicotine Agents Against Cardiovascular Symptoms

This example illustrates the efficacy of anti-nicotine agents as descried herein against effects of cardiovascular disease such as blood pressure, vasoconstriction and heart rate.

Rats (n=4) were administered a single dose of 8D1-IgG4 in the evening of Day 1, 2, 4, and 8, and a single dose of nicotine each morning for 5 days, and six repeated doses on the 9^{th} day, as shown in the table below.

| | | | 8D1-IgG4 plasma | | 8D1-IgG4 |
|---|---|---|---|---|---|
| n = 4 | Nicotine Dose (iv; AM) | | level (AM) | | (iv; PM dose) |
| Day 1 | 0.03 | mg/kg | — | | 20 mg/kg |
| Day 2 | 0.03 | mg/kg | 305 | μg/ml | 40 mg/kg |
| Day 3 | 0.03 | mg/kg | 770 | μg/ml | — |
| Day 4 | 0.03 | mg/kg | 550 | μg/ml | 40 mg/kg |
| Day 5 | 0.03 | mg/kg | 885 | μg/ml | — |
| Day 6-7 | — | | — | | — |
| Day 8 | — | | — | | 80 mg/kg |
| Day 9 | 6 × 0.03 | mg/kg | 1635 | μg/ml | — |

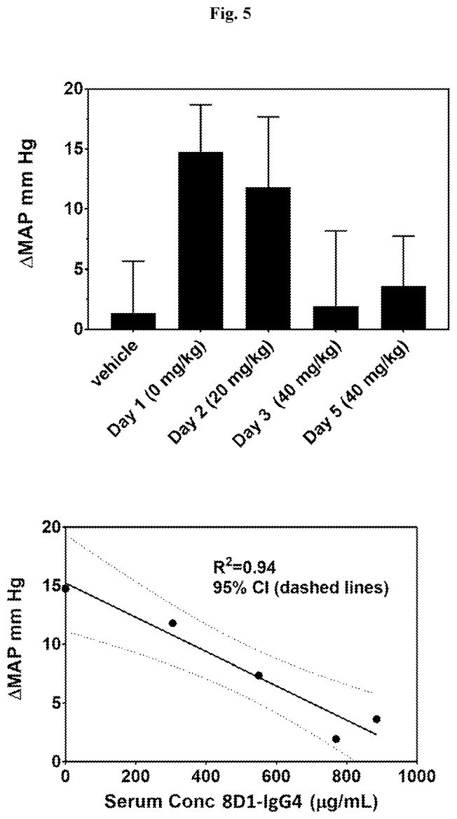

Mean arterial pressure (MAP was taken 2 minutes immediately before and after nicotine administration. The average change in MAP from the two minutes before and after nicotine administration and the correlation between this change in MAP and the plasma concentration of the antibody are shown in FIG. 5. These data indicate that the antibody significantly blocks nicotine-induced increases in MAP in vivo.

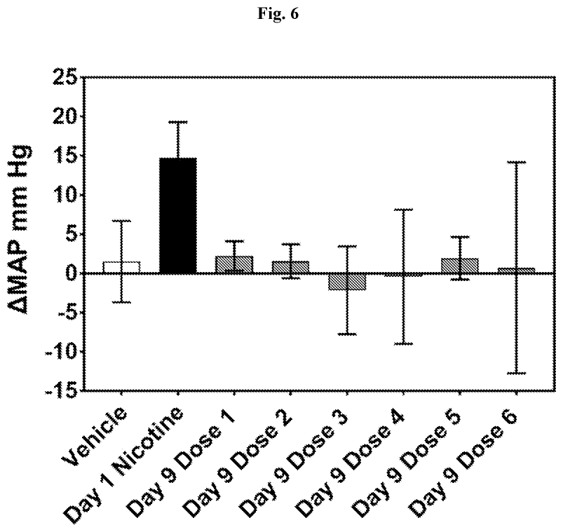

These effects remained constant even with repeated administration of nicotine. For example, 80 mg/kg 8D1-IgG4 was administered intravenously in the evening before challenge with nicotine. The next day, the same rat was administered 6 nicotine doses (0.03 mg/kg, iv) once an hour for 5 h to simulate heavy smoking (equivalent to 12 cigs). The difference in MAP was measure by taking measurements 2 minutes before vs. 2 minutes after each nicotine dose. As shown in FIG. 6, there was very little increase in MAP even after 6 doses of nicotine.

### Example 3—Treatment of Buerger's Disease

This example illustrates methods using anti-nicotine antibodies or nicotine-degrading enzymes in the treatment of Buerger's Disease.

A human subject diagnosed with Buerger's Disease who is a current smoker is administered a therapeutically effective amount of a pharmaceutical composition comprising a nicotine-binding antibody or nicotine-degrading enzyme, by intravenous, intramuscular, or subcutaneous injection. The subject is evaluated for the presence and/or severity of signs and symptoms associated with Buerger's Disease, such as increased blood pressure, vasoconstriction, and increased heart rate, and the subject is treated until one or more signs/symptoms is reduced, ameliorated, or eliminated.

### Example 3—Treatment of CLI

This example illustrates methods using anti-nicotine antibodies or nicotine-degrading enzymes in the treatment of CLI.

A human subject diagnosed with CLI who is a current smoker is administered a therapeutically effective amount of a pharmaceutical composition comprising a nicotine-binding antibody or nicotine-degrading enzyme, by intravenous, intramuscular, or subcutaneous injection. The subject is evaluated for the presence and/or severity of signs and symptoms associated with CLI, such as tissue damage, pain, poorly healing ulceration, gangrene, and loss of digits, and the subject is treated until one or more signs/symptoms is reduced, ameliorated, or eliminated.

1-9. (canceled)

10. A method of improving circulation or treating a cardiovascular disease in a subject in need thereof, comprising administering to the subject a therapeutically effective amount of an anti-nicotine agent.

11. The method of claim 10, wherein the anti-nicotine agent is a nicotine-binding antibody or nicotine-binding fragment thereof.

12. The method of claim 10, wherein the anti-nicotine agent is a nicotine-degrading enzyme.

13. The method of claim 10, wherein the anti-nicotine agent is administered by a route of administration selected from intravenously, subcutaneously, intramuscularly, and intraperitoneally.

14. The method of claim 10, wherein the subject is a current consumer of a nicotine product.

15. The method of claim 14, wherein the subject is an active smoker, e-cigarette user, or tobacco user.

16. The method of claim 10, wherein the subject is undergoing smoking cessation therapy.

17. The method of claim 10, wherein the subject has reduced consumption of a nicotine product.

18. The method of claim 10, wherein the subject is abstaining from consumption of a nicotine product.

19. The method of claim 18, wherein the subject is abstaining from one or more of smoking, e-cigarette use, or tobacco use.

20. The method of claim 18, wherein the abstaining subject is at risk of relapsing.

21. The method of claim 10, wherein the method is effective to reduce one or more symptoms of cardiovascular disease in the subject selected from blood pressure, vasoconstriction, and heart rate.

22. The method of claim 10, wherein the cardiovascular disease is selected from one or more of Buerger's Disease, critical limb ischemia, peripheral artery disease, thrombus formation, and atherosclerotic disease.

23. The method of claim 22, wherein the atherosclerotic disease comprises atherosclerosis that affects the heart or brain.

24. The method of claim 22, wherein the cardiovascular disease is Buerger's Disease.
